SEQUENCE LISTING (1 sequence)

SEQ ID NO: 1 - protein, 368 aa, Homo sapiens
MNPAAEAEFNILLATDSYKVTHYKQYPPNTSKVYSYFECREKKTENSKLRKVKYEETVFYGLQYILNKYLKGKVVTKEKIQEAKDVYKEHFQDDVFNEKGWNYILEKYDGHLPIEIKAVPEGFVIPRGNVLFTVENTDPECYWLTNWIETILVQSWYPITVATNSREQKKILAKYLLETSGNLDGLEYKLHDFGYRGVSSQETAGIGASAHLVNFKGTDTVAGLALIKKYYGTKDPVPGYSVPAAEHSTITAWGKDHEKDAFEHIVTQFSSVPVSVVSDSYDIYNACEKIWGEDLRHLIVSRSTQAPLIIRPDSGNPLDTVLKVLEILGKKFPVTENSKGYKLLPPYLRVIQGDGVDINTLQEVCVLY

(19) United States
(12) Patent Application Publication
(10) Pub. No.: US 2009/0010876 A1
(43) Pub. Date: Jan. 8, 2009

(54) VISFATIN AND USES THEREOF

(75) Inventors: Ira Tabas, New City, NY (US); Yankun Li, Danbury, CT (US)

Correspondence Address: WilmerHale/Columbia University, 399 PARK AVENUE, NEW YORK, NY, 10022, US

(73) Assignee: THE TRUSTEES OF COLUMBIA UNIVERSITY IN THE CITY OF NEW YORK, New York, NY (US)

(21) Appl. No.: 12/123,988

(22) Filed: May 20, 2008

Related U.S. Application Data
(60) Provisional application No. 60/939,234, filed on May 21, 2007.

(51) Int. Cl. A61K 38/20; A61K 38/45; A61K 31/7052; A61K 38/16; A61P 9/00; A61K 38/08; A61K 39/395

(52) U.S. Cl. 424/85.2; 424/94.5; 514/44; 514/15; 424/130.1; 514/12

(57) ABSTRACT

The invention is directed to methods for treating, inhibiting or prevent the incidence of vascular disease in a subject or in a non-human animal by administering visfatin. Particularly, the invention provides for methods to prevent phagocyte cell death due to ER-stress by the administration of visfatin. The invention also encompasses methods for identifying a visfatin polypeptide or a visfatin nucleic acid capable of treating a vascular disease as well as pharmaceutical compositions comprising visfatin.

This application claims the benefit of and priority to U.S. provisional patent application Ser. No. 60/939,234, filed May 21, 2007, the disclosure of all of which is hereby incorporated by reference in its entirety for all purposes.

This invention was made with government support under W81XWH-06-1-0212 awarded by US Army Medical Research and Materiel Command (USAMRMC). The government has certain rights in the invention.

### BACKGROUND OF THE INVENTION

As of 1999, an estimated 12.6 million Americans had coronary heart disease and approximately 1 in 5 deaths in 1999 were due to coronary disease complications. In addition to the social burden of the disease, the economic consequences of coronary heart disease are manifested as a direct cost to our health care system as well as a cost associated with premature and permanent disability of the labor force. Atherosclerosis, a major contributor to morbidity and mortality associated with heart disease is the process in which fatty deposits, cholesterol and other cellular products accumulate on the arterial lining to form plaques. As plaques increase in size, plaque rupture can result in the formation of blood clots and if the clot moves to the heart, lungs, or brain, it can cause a heart attack, or pulmonary embolism or stroke.

An important event in the progression of atherosclerotic lesions is the accumulation of macrophages in the subendothelial space of activated luminal endothelium. Once in this atherogenic lipoprotein rich environment, macrophages ingest large quantities of lipids and cholesterol and convert into foam cells as a result of their accumulation in intracellular compartments. The toxicity associated with the excessive uptake of such compounds manifests itself in the activation of the a cellular stress pathway called the unfolded protein response (UPR) pathway. Important in several biological contexts, activation of the UPR in macrophages in atherosclerotic plaques contributes to their death and to the eventually formation of a necrotic core. These necrotic cores in turn can contribute to thrombotic events leading to ischemia, cardiac failure and stroke.

### SUMMARY OF THE INVENTION

The invention relates to visfatin and the pharmaceutically acceptable derivatives thereof, and to their use alone or in combination with other active agents for preventing, halting or slowing the progression of atherosclerosis and related conditions and disease events.

In one aspect, the invention provides methods to treat diseases involving macrophage cell death. The invention is based on the discovery that there are at least two actions of visfatin (or bioactivity, or effects of visfatin) in macrophages that have not previously been reported: (1) suppression of macrophage cell death and (2) suppression of a cell stress pathway called the unfolded protein response (UPR). Macrophage cell death has been implicated in the progression of atherothrombotic disease, so blocking this event may prevent atherothrombotic vascular disease. The UPR has been implicated in insulin resistance and type 2 diabetes, and so blocking this pathway may be beneficial in this setting. New therapeutic strategies using compounds that mimic visfatin activity or that can increase or enhance visfatin activity or effect are encompassed by this invention. The methods of this invention which are useful to block macrophage cell death and the UPR are methods which can be used to treat or inhibit heart disease or diabetes and other diseases.

The invention provides a method for treating a vascular disease in a subject, the method comprising administering to the subject a pharmaceutically effective amount of a visfatin polypeptide or a visfatin nucleic acid to increase visfatin activity.

The invention also provides a method for inhibiting the development of a vascular disease in a subject, the method comprising administering to the subject a pharmaceutically effective amount of a visfatin polypeptide or a visfatin nucleic acid to increase visfatin activity.

Further provided for by the invention is a method for treating a subject at risk of vascular disease, the method comprising administering to the subject a pharmaceutically effective amount of a visfatin polypeptide or a visfatin nucleic acid to increase visfatin activity.

An aspect of the invention provides an in-vivo method for identifying a visfatin polypeptide or a visfatin nucleic acid capable of treating a vascular disease, the method comprising: (a) administering a pharmaceutically effective amount of a visfatin polypeptide or a visfatin nucleic acid to a first non-human transgenic animal; (b) measuring an incidence of atherosclerotic plaque formation in the first non-human transgenic animal; and (c) comparing the measured incidence of atherosclerotic plaque formation in the first non-human transgenic animal to a measured incidence of atherosclerotic plaque formation in a genetically similar second transgenic non-human animal that has not been administered a visfatin polypeptide or a visfatin nucleic acid, wherein a decrease the in number, size, or susceptibility to rupture of the atherosclerotic plaques in the first non-human transgenic animal compared to the second non-human transgenic animal indicates that the visfatin polypeptide or the visfatin nucleic acid is capable of treating vascular disease. In one embodiment, the non-human transgenic animal has a genetic modification. In another embodiment, the genetic modification of the transgenic animal results in a greater incidence of vascular disease compared to a genetically similar wild-type non-human animal. In yet another embodiment, the genetic modification of the non-human transgenic animal comprises a transgenic modification to increase of apoB gene expression, a transgenic modification resulting in a loss of apolipoprotein E gene function, a transgenic modification resulting in a loss of LDL receptor gene function, a transgenic modification resulting in a loss of eNOS gene function, a transgenic modification resulting in a loss of apoBEC gene function or any combination thereof. In a further embodiment, the method comprises an additional step of causing a vascular injury. In one embodiment, the vascular injury comprises, a wire-induced injury, a carotid artery ligation-induced vascular injury, an electric current-induced vascular injury, a perivascular collar-induced vascular injury, a vein graft-induced vascular injury, or an allograft-induced vascular injury, or any combination thereof.

In a further embodiment, the in-vivo method for identifying a visfatin polypeptide or a visfatin nucleic acid capable of treating a vascular disease of the invention comprises an additional step of feeding the non-human animal a diet that can promote the incidence of atherosclerosis. In another embodiment, the diet that can promote the incidence of atherosclerosis in the in-vivo method for identifying a visfatin polypeptide or a visfatin nucleic acid capable of treating a vascular disease of the invention comprises a high cholesterol diet, a high fat diet, a high fat western diet or any combination thereof. In yet a further embodiment, the in-vivo method for identifying a visfatin polypeptide or a visfatin nucleic acid capable of treating a vascular disease of the invention comprises an additional step of administering streptozotocin.

In a further embodiment of the invention, the incidence of atherosclerotic plaque formation is assessed by quantification of aortic root lesion area, en-face aortic lesion area analysis, analysis of the cellular composition of lesions, quantification of markers of lesion progression, assessment of apoptosis, assessment of the expression of CHOP and other UPR markers in lesional cells, or quantification of gene expression using laser-capture microdissection or any combination thereof.

Another aspect of the invention provides for an in-vivo method for identifying a visfatin polypeptide or a visfatin nucleic acid capable of treating a vascular disease, the method comprising: (a) subjecting a first non-human animal to a condition capable of causing vascular disease; (b) administering a pharmaceutically effective amount of a visfatin polypeptide or a visfatin nucleic acid to the first non-human animal; (c) measuring an incidence of atherosclerotic plaque formation in the first non-human animal; and (d) comparing the measured incidence of atherosclerotic plaque formation in the first non-human animal to a measured incidence of atherosclerotic plaque formation in a genetically similar second non-human animal that has not been administered a visfatin polypeptide or a visfatin nucleic acid, wherein a decrease the in number, size or susceptibility to rupture of the atherosclerotic plaques in the first non-human animal compared to the second non-human animal indicates that the visfatin polypeptide or the visfatin nucleic acid is capable of treating vascular disease. In accordance with methods of the invention, the condition capable of causing vascular disease comprises: a condition of hypercholesterolemia, a condition of hyperlipoproteinemia, a condition of hypertriglyceridemia, a condition of lipodystrophy, a condition of hyperglycemia, a condition of reduced HDL levels, a condition of elevated LDL levels, a condition of low glucose tolerance, a condition of insulin resistance, a condition of obesity, a condition of dyslipidemia, a condition of hyperlipidemia, a condition of hypercholesterolemia, a condition of vascular restenosis, a condition of hypertension, a condition of Type I diabetes, a condition of Type II diabetes, a condition of hyperinsulinemia, a condition of atherogenesis, a condition of angina, a condition of ischemic heart disease, an aneurysm, a neointimal hyperplasia following percutaneous a transluminal coronary angiograph, a vascular graft, a coronary artery bypass surgery, a thromboembolic event, a post-angioplasty restenosis, a coronary plaque inflammation, an embolism, a stroke, an arrhythmia, an atrial fibrillation or atrial flutter, a thrombotic occlusion, a high cholesterol diet, a high fat diet, or a high fat Western diet or any combination thereof.

In yet another aspect, the invention provides a method for preventing phagocyte death in a subject, the method comprising administering to the subject a pharmaceutically effective amount of a visfatin polypeptide or a visfatin nucleic acid to increase visfatin activity

In another aspect, the invention provides a method for preventing plaque necrosis in a subject, the method comprising administering to the subject a pharmaceutically effective amount of a visfatin polypeptide or a visfatin nucleic acid to increase visfatin activity.

In accordance with methods of the invention, visfatin activity comprises: suppression of unfolded protein response (UPR) pathway activation, ERK activation, AKT activation, protection of phagocytes from endoplasmic reticulum (ER) stress mediated cell death, suppression of UPR activation induced production of CHOP, suppression of UPR activation induced production of ATF3, suppression of UPR activation induced production of ATF4, suppression of UPR activation induced production of XBP1, phosphorylation of insulin receptor substrate-2 (IRS2), or cytoplasmic FOXO1 or any combination thereof.

Also in accordance with methods of the invention, vascular disease comprises: an advanced atherosclerotic lesion, atherosclerosis, arteriosclerosis, thrombosis, restenosis, hypertension, angina pectoris, arrhythmia, heart failure, myocardial infarction, thrombosis, thromboembolytic stroke, peripheral vascular diseases, cerebral ischemia, or cardiomyopathy, or any combination thereof.

In some embodiments of the invention, the vascular disease comprises formation of a necrotic core at the site of an atherosclerotic plaque. In other embodiments of invention; the formation of the necrotic core comprises phagocyte death. In the embodiments, the formation of the necrotic core comprises a reduced clearance of dead phagocytes. In yet other embodiments, the phagocyte death occurs by apoptosis, necrosis, autophagy, oncolysis, or mitoptosis, or any combination thereof. In further embodiments, the vascular disease is caused by an impairment of phagocyte function. In some embodiments, the impairment of phagocyte function comprises an uptake of excessive cholesterol, uptake of excessive oxidized LDL, uptake of excessive acetylated LDL, reduced cholesterol esterification, a defect in lipid trafficking, a defect in protein trafficking, intracellular cholesterol accumulation, conversion to a foam cell morphology, phagocyte apoptosis, phagocyte necrosis and defective ingestion of dead cells. In other embodiments, the impairment of phagocyte function is associated with an activation of the UPR pathway. In yet other embodiments, the activation of the UPR pathway is due to ER stress. In some embodiments, the activation of the UPR pathway is due to an enrichment of free cholesterol in the ER membranes of the phagocyte. In other embodiments, the activation of the UPR pathway is caused by an oxidized lipid, celecoxib, fenofibrate, homocysteine, hypoxia, or insulin resistance or any combination thereof.

In some embodiments of the invention, the phagocyte is selected from the group consisting of a microglial cell, a monocyte, a microglial precursor cell, a monocyte precursor cell, a macrophage precursor cell, a microglial-like cell, a monocyte-like cell, a dendritic-like cell, and a macrophage-like cell. In other embodiments of the invention, the phagocyte is a macrophage or a derivative thereof.

In accord with this invention, the visfatin polypeptides of the invention comprise: a visfatin polypeptide, a peptidomimetic agent, a truncation product of a visfatin polypeptide, a fragment of a visfatin polypeptide, a polypeptide that is homologous to visfatin, a polypeptide of SEQ ID NO:1 or a polypeptide having a sequence at least 85% identical to the amino acid sequence in SEQ ID NO:1 and exhibiting visfatin activity. In some embodiments, the visfatin polypeptide has at least 99%, 97%, 95%, 90%, 80% or 70% amino acid sequence identity to the amino acid sequence in SEQ ID NO:1.

Also in accordance with the invention, the visfatin nucleic acids of the invention comprise: a nucleic acid molecule that can encode a visfatin polypeptide, a nucleic acid molecule that can encode a peptidomimetic agent of a visfatin polypeptide, a nucleic acid molecule that can encode a truncation product of a visfatin polypeptide, a nucleic acid molecule that can encode a fragment of a visfatin polypeptide, a nucleic acid molecule that can encode a polypeptide that is homologous to visfatin, a nucleic acid molecule that can encode a polypeptide of SEQ ID NO:1 or a nucleic acid molecule that can encode polypeptide having a sequence at least 85% identical to the amino acid sequence in SEQ ID NO:1 and exhibiting visfatin activity. In some embodiments, the nucleic acid molecule that can encode a visfatin polypeptide that has at least 99%, 97%, 95%, 90%, 80% or 70% amino acid sequence identity to the amino acid sequence in SEQ ID NO:1.

In some embodiments, the method further comprises an additional step of administering of one or more additional therapeutic agents. In other embodiments, the one or more additional therapeutic agents are capable of inhibiting UPR-induced cell death. In yet other embodiments, the one or more additional therapeutic agents are selected from the group comprising: p38 MAPK inhibitors, including SB202190, PD169316, FR167653. SB203580, ARRY-797, SB 239063, SC-68376, SB 220025, SB-200646, PD 169316 or SKF-86002; p38 substrate peptides; JNK2 inhibitors, including, SP600125, a polypeptide comprising residues 153-163 of JNK-interacting protein-1 (JIP-1), AS601245 or N-(4-Amino-5-cyano-6-ethoxypyridin-2-yl)-2-(2,5-dimethoxyphenyl)acetamide; or SRA inhibitors, including SRA blocking antibodies. In yet another embodiment, the one or more additional therapeutic agents are capable of activating Stat3. In still other embodiments, the one or more additional therapeutic agents are capable of activating Stat3 are selected from the group comprising: IL-1, IL-6, IL-22, VEGF, leptin, bFGF, LIF, EGF, NRG-1, GH, IL-4, CNTF, or PIF or any combination thereof. In one embodiment, the additional therapeutic agent is IL-10. In other embodiments, the one or more additional therapeutic agents are selected from the group comprising: lipoxin, a lipoxin analog, or a compound that stimulates lipoxin synthesis or activity, a statin, a beta-blocker, a thiozide diuretic, an angiotensin-converting enzyme inhibitor, omega-3 fatty acids, aspirin, clopidogrel, an aldosterone agonist, nitrates, calcium channel blockers, cholesterol-uptake inhibitors; cholesterol biosynthesis inhibitors, including HMG-CoA reductase inhibitors or statins; HMG-CoA synthase inhibitors; squalene epoxidase inhibitors and squalene synthetase inhibitors; acyl-coenzyme A cholesterol acyltransferase (ACAT) inhibitors, including, melinamide; probucol; 58035; nicotinic acid and salts thereof; niacinamide; cholesterol absorption inhibitors, including, beta-sitosterol and ezetimibe; bile acid sequestrant anion exchange resins, including cholestyramine, colestipol, colesevelam and dialkylaminoalkyl derivatives of a cross-linked dextran; LDL receptor ligands; fibrates, including clofibrate, bezafibrate, fenofibrate and gemfibrozil; vitamin B6 and pharmaceutically acceptable salts thereof; vitamin B12, including cyanocobalamin and hydroxocobalamin; vitamin B3; anti-oxidant vitamins, including vitamin C, vitamin E, and betacarotene; angiotensin II receptor antagonists; renin inhibitors; platelet aggregation inhibitors, including fibrinogen receptor antagonists; estrogen, insulin, benfluorex; ethyl icosapentate; amlodipine, U18666A, celecoxib, fenofibrate, an SRA blocking antibody, anti-inflammatory agents or anti-arrhythmic agents or any combination thereof. In another embodiment, the one or more additional therapeutic agents is a siRNA, a microRNA, an aptamer, or an antibody.

In embodiments provided by the invention, the visfatin polypeptide, the nucleic acid that can encode a visfatin polypeptide or the additional therapeutic agent is administered via an osmotic pump. In some embodiments, the administering is carried out orally, rectally, parenterally, subcutaneously, intramyocardially, transendocardially, transepicardially, topically, intravenously, intramuscularly, intraperitoneally, intraarterially, transdermally, endoscopically, intralesionally, percutaneously, intrathecally or by application to mucous membranes, such as, that of the nose, throat, and bronchial tubes.

One aspect of the invention provides a method for determining whether a compound is capable enhancing the ability of visfatin to suppress activation of the UPR pathway, the method comprising: (a) contacting a first cell with an agent that induces ER stress; (b) contacting the first cell with an amount of visfatin polypeptide; (c) measuring an indicator of UPR pathway activation in the first cell; and (d) comparing the indicator of UPR pathway activation with a second cell that has been subjected to an additional step of being contacted with a test compound, wherein a decrease in the measured indicator of the UPR pathway activation in the second cell compared to the first cell indicates that the compound is capable of enhancing the ability of visfatin to suppress activation of the UPR pathway.

Another aspect of the invention provides a method for identifying a mimetic of visfatin, the method comprising: (a) contacting a first cell with an agent that induces ER stress; (b) measuring an indicator of UPR pathway activation in the first cell; and (c) comparing the indicator of UPR pathway activation with a second cell that has been contacted with a test compound, wherein a decrease in the measured indicator of the UPR pathway activation in the second cell compared to the first cell indicates that the compound is a mimetic of visfatin.

Also provided by the invention is a method for identifying a compound that is capable of inducing the production of visfatin, the method comprising: (a) contacting a cell with an agent that induces ER stress; (b) contacting the cell with an amount of visfatin polypeptide; (c) measuring an indicator of UPR pathway activation in the first cell; and (d) comparing the measured indicator of UPR pathway activation in a second cell that has been subjected to an additional step of being contacted with a test compound, wherein a decrease in the measured indicator of UPR pathway activation in the second cell compared to the first cell indicates that the compound is capable enhancing the ability of visfatin to suppress activation of the UPR pathway.

In yet another aspect, the invention provides a method for identifying a cell that is capable of responding to visfatin, the method comprising: (a) contacting a first cell with an agent that induces ER stress; (b) contacting the first cell with an amount of visfatin polypeptide; (c) measuring an indicator of UPR pathway activation in the first cell; and (d) comparing the measured indicator of UPR pathway activation in a genetically similar second cell that has been contacted with an agent that induces ER stress but has not been contacted with visfatin, wherein a decrease in the measured indicator of the UPR pathway activation in the first cell compared to the second cell indicates that the first cell is capable of responding to visfatin.

In some embodiments of the invention, the methods of the invention provide for an additional step of contacting the first cell or the second cell with an agent that promotes cell death under conditions of ER stress. In other embodiments, the agent that promotes cell death under conditions of ER stress is an SRA ligand. In some embodiments, the SRA ligand is selected form the group comprising: fucoidan, cholesterol saturated methyl-beta cyclodextrin or carboxymethyllysine BSA. In yet other embodiments, the agent that promotes cell death under conditions of ER stress is an activator of p38/MAPK. In other embodiments, the agent that promotes cell death under conditions of ER stress is and activator of JNK2. In accordance with methods of the invention, the agent that induces ER stress is any of: protein glycosylation inhibitors, including tunicamycin; sarcoendoplasmic reticulum calcium ATPase inhibitors, including, thapsigargin, calcium ionophores, including A23187, agents that increase intracellular cholesterol, including free cholesterol, oxidized cholesterol, oxidized LDL, acetylated LDL, lipopolysaccharide, Brefeldin A, celecoxib, fenofibrate, homocysteine, or Dithiothreitol, or any combination thereof.

Some embodiments of the invention provide for measuring of the indicator of UPR pathway activation by measuring formation of an antibody-antigen complex. In yet other embodiments, the formation of antigen-antibody complex is detected by immunoassay based on Western blot technique, ELISA, indirect immunofluorescence assay, or immunoprecipitation assay, wherein the immunoassay is used to detect any of: ATF4 expression, ATF3 expression, GADD34 expression, CHOP expression, XBP1 expression, dissociation of BiP and PERK, dissociation of BiP and IRE1 ATF4 nuclear translocation, pATF(N) nuclear translocation, pAFT6 translocation to the Golgi apparatus, pATF6 proteolytic cleavage. PERK phosphorylation, IRE1 phosphorylation, an increase in the production of ER chaperones, an increase in the production of ER folding enzymes, or any combination thereof.

In some embodiments of the invention, measuring the indicator of UPR pathway activation involves measuring an amount of a cellular RNA. In other embodiments, the amount of a cellular RNA is detected by an amplification or hybridization assay. In yet other embodiments, the amplification assay is quantitative or semiquantitative PCR. In further embodiments, the hybridization assay is selected from the group consisting of Northern blot, dot or slot blot, nuclease protection and microarray assays. In some embodiments of the invention, the amplification or hybridization assay is used to detect cellular RNA for any of: ATF4, ATF3, GADD34, CHOP, XBP1, an ER chaperone, an ER folding enzyme, or any combination thereof.

In other embodiments of the invention, measuring the indicator of UPR pathway activation involves measuring an amount of cell death. In further embodiments, measuring the amount of cell death involves measuring the formation of an antibody-antigen complex. In accordance with methods of the invention, the antibody is used to measure cell death is specific for a protein selected from the croup consisting of phospho-histone H3, phosphorylated MAP kinase, phosphorylated MEK-1, BM28, cyclin E, p53, Rb and PCNA. In yet other embodiments, the measuring of cell death is performed using flow cytometry. In further embodiments provided by the invention, the measuring of cell death is performed using apoptosis markers. In some embodiments, the apoptosis marker is selected from the group consisting of Annexin V, TUNEL Stain, 7-amino-actinomycin D and Caspase substrates.

One aspect of the invention provides a method for treating an ER stress-related disease in a subject, the method comprising administering to the subject a pharmaceutically effective amount of a visfatin polypeptide or a visfatin nucleic acid to increase visfatin activity.

Another aspect of the invention provides a method for inhibiting the development of an ER stress-related disease in a subject, the method comprising administering to the subject a pharmaceutically effective amount of a visfatin polypeptide or a visfatin nucleic acid to increase visfatin activity.

Also provided by the invention is a method for treating a subject at risk of an ER stress-related disease, the method comprising administering to the subject a pharmaceutically effective amount of a visfatin polypeptide or a visfatin nucleic acid to increase visfatin activity.

In accordance with the methods of the invention, the ER stress-related disease comprises: Alzheimer's disease, Parkinson's disease, Huntington's disease, spinobulbar muscular atrophy (Kennedy disease), Machado-Joseph disease, dentatorubral-pallidoluysian disease (Haw River Syndrome), spinocerebellar ataxia, Pelizaeus-Merzbacher disease, Prion disease, Creutzfeldt-Jakob disease, Gertsmann-Straussler-Scheinker syndrome, fatal familial insomnia, Kuru, Alpers syndrome, bovine spongiform encephalopathy, transmissible milk encephalopathy, chronic wasting disease, scrapie, amyotrophic lateral sclerosis (Lou Gehrig's disease), GM1 gangliosidosis, bipolar disorders, type I diabetes mellitus, type II diabetes mellitus, Walcott-Rallison syndrome or hereditary tyrosinemia type I, or any combination thereof.

In one aspect, the invention provides for a pharmaceutical composition comprising a visfatin polypeptide, in admixture with a pharmaceutically acceptable adjuvant, diluent or carrier.

In another aspect, the invention provides for a pharmaceutical composition comprising (i) a visfatin polypeptide, in admixture with a pharmaceutically acceptable adjuvant, diluent or carrier, diluent or carrier; and (ii) a cholesterol-lowering agent, in admixture with a pharmaceutically acceptable adjuvant, diluent or carrier; wherein the visfatin polypeptide and the cholesterol-lowering agent are each provided in a form that is suitable for administration in conjunction with the other.

In a further aspect, the invention provides for a pharmaceutical composition comprising (i) a visfatin polypeptide, in admixture with a pharmaceutically acceptable adjuvant, diluent or carrier, diluent or carrier; and (ii) a beta blocker, in admixture with a pharmaceutically acceptable adjuvant, diluent or carrier; wherein the visfatin polypeptide and the beta blocker are each provided in a form that is suitable for administration in conjunction with the other.

In yet another aspect, the invention provides for a pharmaceutical composition comprising (i) a visfatin polypeptide, in admixture with a pharmaceutically acceptable adjuvant, diluent or carrier, diluent or carrier; and (ii) an anti-inflammatory agent, in admixture with a pharmaceutically acceptable adjuvant, diluent or carrier; wherein the visfatin polypeptide and the anti-inflammatory agent are each provided in a form that is suitable for administration in conjunction with the other.

In another aspect, the invention provides for a pharmaceutical composition comprising (i) a visfatin polypeptide, in admixture with a pharmaceutically acceptable adjuvant, diluent or carrier, diluent or carrier; and (ii) a cholesterol-lowering agent, in admixture with a pharmaceutically acceptable adjuvant, diluent or carrier; (iii) a beta blocker, in admixture with a pharmaceutically acceptable adjuvant, diluent or carrier; (iv) a anti-inflammatory agent, in admixture with a pharmaceutically acceptable adjuvant, diluent or carrier wherein the visfatin polypeptide, the beta blocker, the cholesterol lowering agent and the anti-inflammatory agent are each provided in a form that is suitable for administration in conjunction with the other pharmaceutical formulations.

### BRIEF DESCRIPTION OF DRAWINGS

FIG. 1. Visfatin protects macrophages from ER stress-mediated apoptosis. FIG. 1A. Macrophages were pre-incubated 1100 ng/ml visfatin (vis) for 24 h and then incubated with 50 μg/ml acetyl-LDL+10 μg/ml 58035 (FC) for 20 h. Apoptosis was assayed by Alexa-488 annexin V staining (green). One representative image per condition was shown. FIG. 1B. The percentage of apoptosis was quantified (mean±SEM, n=8). FIG. 1C. Macrophages incubated in medium (con) or with visfatin (vis) for 24 h. Whole cell lysates were immunoblotted for the insulin receptor β (IRβ), SR-A, CD36 and β-actin. FIG. 1D. Macrophages were pre-incubated ±100 ng/ml visfatin (vis) for 24 h and then incubated with 5 μg/ml tunicamycin +25 μg/ml fucoidan (TN/F) for 24 h. Apoptosis was assayed and quantified as described in FIG. 1A and FIG. 1B.

FIG. 2. Visfatin suppresses the distal UPR activation. FIG. 2A. Macrophages were preincubated 1100 ng/ml visfatin (vis) for 24 h and then incubated with FC, 5 μM thapsigargin (TG), or 5 μg/ml tunicamycin (TN) for 5 h. Whole cell lysates were immunoblotted for CHOP, ATF3, and β-actin. FIG. 2B. Macrophages were pre-incubated ±100 ng/ml visfatin for 24 h and then incubated with 5 μg/ml TN for 5 h. The nuclear extracts were immunoblotted for XBP-1, ATF4, and nucleophosmin (loading control). FIG. 2C. Nuclei-free cell extracts from the above experiment were immunoblotted with antibodies against PERK, IRE1α, phospho-eIf2α, and β-actin. Phosphorylation of PERK and IRE1α was demonstrated by a subtle retardation of migration.

FIG. 3. Visfatin suppresses ATF4 protein synthesis. FIG. 3A. Macrophages were preincubated ±1100 ng/ml visfatin (v) for 24 h and then incubated with 50 μg/ml acetyl-LDL+10 μg/ml 58035 (FC) for indicated times. Total RNA was extracted and subjected to RT-QPCR. The mRNA level of ATF4 was normalized with a control gene 36B4. FIG. 3B. Macrophages were pre-incubated for 24 h±100 ng/ml visfatin (vis) and then incubated for 5 h+5 μg/ml tunicamycin (TN). The cells were pulsed with [35S]methionine/cysteine in methionine-free medium for 15 min and then lysed in RIPA buffer. ATF4 was immunoprecipitated using a rabbit antibody immobilized to protein A/G beads. A normal rabbit IgG served as control. The immunoprecipitate was subjected to SDS-PAGE and autoradiography.

FIG. 4. The UPR-suppressing effect of visfatin is independent of the insulin receptor, macrophages from insulin receptor deficient mice (IR−/−) or littermate wild type control (wt) were preincubated ±100 ng/ml visfatin (v) for 24 h and then incubated with 5 μg/ml tunicamycin (TN) for 5 h. Whole cell lysates were immunoblotted for CHOP and β-actin.

FIG. 5. Recombinant visfatin suppresses the acute UPR induction of macrophages in mouse peritoneal cavity. FIG. 5A. Male C57/B6 mice were subjected to intra-peritoneal (i.p.) injection of 1 ml of 4% thioglycolate to elicit peritoneal macrophages. Three days later, the mice were injected i.v. with 0.1 ml sterile saline ±5 mg recombinant visfatin (vis). Twenty-four hours later, these mice were injected i.p. with 0.5 ml 150 mM dextrose ±25 μg tunicamycin (TN). Twelve hours after TN injection, macrophages were harvested by peritoneal lavage and immediately lysed in RIPA buffer. The lysates were subjected to SDS-PAGE and immunoblotting for CHOP, phospho-eIf2α and β-actin. FIG. 5B. The intensity of CHOP bands were quantified and normalized against that of phospho-eIf2α.

FIG. 6. ATF4 is the direct target of visfatin in macrophages. This is a proposed mechanism and it is included as a possible mechanism of visfatin. This example is not limiting and other mechanisms may be possible.

FIG. 7. Polypeptide sequence of visfatin (SEQ ID NO: 1)

### DETAILED DESCRIPTION OF THE INVENTION

One of the early events in atherosclerosis is the entry of monocytes into focal areas of the arterial subendothelium that have accumulated matrix-retained lipoproteins and modified lipoproteins. These monocytes differentiate into macrophages and the macrophages accumulate large amounts of intracellular cholesterol through the ingestion of lipoproteins in the subendothelium. Upon ingestion, the cholesterol is stored in an esterified form within subcellular lipid vesicles. As the macrophages continue to ingest cholesterol, the cellular mechanisms that function to maintain a cholesterol homeostasis fail and the macrophages become loaded with excess free cholesterol and adopt a foam cell morphology. In advanced atherosclerotic lesions, accumulation of large amounts of free cholesterol (FC) within lesional macrophages induces macrophage apoptosis due to free cholesterol-induced toxicity. This is speculated to contribute to plaque instability. Unlike cholesterol esters, free cholesterol can insert into lipid bilayers and alter the physical properties of biological membranes. In macrophages, the accumulation of excess free cholesterol results in cholesterol loading of the endoplasmic reticulum (ER), the depletion of ER calcium store, activation of the unfolded protein response (UPR) and eventually in cell death. Activation of the UPR pathway is a key event in FC-induced apoptosis.

As used herein and in the appended claims, the singular forms “a,” “an,” and “the” include plural references unless the content clearly dictates otherwise. Thus, for example, reference to “a compound” includes a plurality of such agents and equivalents thereof known to those skilled in the art, and reference to “visfatin” is a reference to one or more visfatin polypeptides and equivalents thereof known to those skilled in the art, and so forth. All publications, patent applications, patents, and other references mentioned herein are incorporated by reference in their entirety. The patent and scientific literature referred to herein establishes knowledge that is available to those skilled in the art. The issued patents, applications, and other publications that are cited herein are hereby incorporated by reference to the same extent as if each was specifically and individually indicated to be incorporated by reference. In the case of inconsistencies, the present disclosure will prevail.

### DEFINITIONS

The following definitions are presented as an aid in understanding this invention.

As used herein a “Visfatin” or “PBEF” or “Pre-B Cell Colony Enhancing Factor” or “NAMPT” or Nicotinamide Phosphorybosyltransferase” means an adipokine protein, having a molecular weight of about 52,000 daltons. “Visfatin” also refers to the polypeptide having SEQ ID NO:1. The term “visfatin” also refers to other species specific isoforms. For example, the term visfatin encompasses the human isoforms of visfatin having NCBI accession numbers of EAL24400, EAW83384, EAW83383, EAW83382, CAI17061, or NP_{—}005737.

As used herein, “visfatin” also includes a “visfatin protein” and a “visfatin analog” A “visfatin analog” is a functional variant of the visfatin protein, having visfatin biological activity, and that can have 85% or greater amino-acid-sequence identity with the visfatin protein. The visfatin polypeptides of the invention can be unglycosylated or glycosylated. As used herein, “visfatin” also includes mimetics of visfatin.

As further used herein, the term “visfatin activity” means biological activity induced by visfatin. The term “visfatin activity” also refers to the activity of a protein or peptide that demonstrates an ability to suppress UPR activation under a condition of ER stress under the conditions of the assays described herein.

As used herein, the term “nucleic acid molecule” and “polynucleotide” are used interchangeably to refer to a polymeric form of nucleotides of any length, either deoxyribonucleotides or ribonucleotides, or analogs thereof. Polynucleotides may have any three-dimensional structure, and may perform any function, known or unknown. Non limiting examples of polynucleotides include a gene, a gene fragment, exons, introns, messenger RNA (mRNA), transfer RNA, ribosomal RNA, ribozymes, cDNA, recombinant polynucleotides, branched polynucleotides plasmids, vectors, isolated DNA of any sequence, isolated RNA of any sequence, nucleic acid probes, and primers. A polynucleotide is typically composed of a specific sequence of four nucleotide bases: adenine (A); cytosine (C); guanine (G); and thymine (T) or uracil (U). Thus, the term polynucleotide sequence is the alphabetical representation of a polynucleotide molecule. This alphabetical representation can be input into databases in a computer having a central processing unit and used for bioinformatics applications such as functional genomics and homology searching.

In addition to polypeptides consisting only of naturally-occurring amino acids, peptidomimetics are also provided. Peptide analogs are commonly used in the pharmaceutical industry as non-peptide drugs with properties analogous to those of the template peptide. These types of non-peptide compound are termed, “mimetics”, “peptide mimetics” or “peptidomimetics” and are usually developed with the aid of computerized molecular modeling. Eichler et al. (1995) Med. Res. Rev. 15:481-496; Moore et al. (1995) Adv. Pharmacol. 33:1-41; Moore (1994) Trends Pharmacol. Sci. 15:124-129; Saragovi et al. (1992) Biotechnol. 10:773-778. Peptide mimetics that are structurally similar to therapeutically useful peptides can be used to produce an equivalent therapeutic or prophylactic effect. Generally, peptidomimetics are structurally similar to a paradigm polypeptide (i.e., a polypeptide that has a biochemical property or pharmacological activity), such as visfatin, but have one or more peptide linkages optionally replaced by a linkage such as: —CH.sub.2NH—, —CH.sub.2S—, —CH.sub.2-CH.sub.2-, —CH.dbd.CH— (cis and trans), —COCH.sub.2-, —CH(OH)CH.sub.2-, and —CH.sub.2SO—, by methods known in the art. See, for example, Spatola (1983) Chemistry and Biochemistry of Amino Acids, Peptides, and Proteins B. Weinstein eds. Marcel Dekker, New York.

As used herein, a “phagocyte” means a cell that is selected from the group consisting of a microglial cell, a monocyte, a macrophage, a microglial precursor cell, a monocyte precursor cell, a macrophage precursor cell, a microglial-like cell, a monocyte-like cell, and a macrophage-like cell.

“Vascular disease” means disorders and diseases that can be treated and/or prevented by administering an amount of a compound or mixture of compounds to increase visfatin activity. “Vascular disease” comprise, without limitation, hypercholesterolemia, hyperlipoproteinemia, hypertriglyceridemia, lipodystrophy, hyperglycemia, genetic susceptibility, low HDL levels, high LDL levels, low glucose tolerance, insulin resistance, obesity, lipid disorders, dyslipidemia, hyperlipidemia, hypercholesterolemia, vascular restenosis, hypertension, Type I diabetes, Type II diabetes, hyperinsulinemia, atherosclerosis, atherogenesis, angina, aneurysm, ischemic heart disease, platelet aggregation, platelet adhesion, neointimal hyperplasia following percutaneous transluminal coronary angiograph, vascular grafting, coronary artery bypass surgery, thromboembolic events, thombosis, post-angioplasty restenosis, coronary plaque inflammation, embolism, stroke, shock, arrhythmia, atrial fibrillation or atrial flutter, thrombotic occlusion and reclusion cerebrovascular incidents, left ventricular dysfunction, or hypertrophy, or any combination thereof.

“Conditions capable of causing vascular disease” comprise, but are not limited to, a condition of hypercholesterolemia, a condition of hyperlipoproteinemia, a condition of hypertriglyceridemia, a condition of lipodystrophy, a condition of hyperglycemia, a condition of HDL reduced levels, a condition of elevated LDL levels, a condition of low glucose tolerance, a condition of insulin resistance, a condition of obesity, a condition of dyslipidemia, a condition of hyperlipidemia, a condition of hypercholesterolemia, a condition of vascular restenosis, a condition of hypertension, a condition of Type I diabetes, a condition of Type II diabetes, a condition of hyperinsulinemia, a condition of atherogenesis, a condition of angina, a condition of aneurysm, a condition of ischemic heart disease, a neointimal hyperplasia following percutaneous a transluminal coronary angiograph, a vascular graft, a coronary artery bypass surgery, a thromboembolic event, a post-angioplasty restenosis, a coronary plaque inflammation, an embolism, a stroke, an arrhythmia, an atrial fibrillation or atrial flutter, a thrombotic occlusion, a high cholesterol diet, a high fat diet, or a high fat western diet or any combination thereof.

An increased “susceptibility to rupture” of an atherosclerotic plaque is defined as increased plaque necrosis, increased inflammation at site of an atherosclerotic plaque, or a thinning of the fibrous cap of an atherosclerotic plaque, or any combination thereof.

A “pharmaceutically effective amount” is any amount of an agent which, when administered to a subject suffering from a disorder against which the agent is effective, causes reduction, remission or regression or prevents recurrence of the disorder.

A “prophylactically effective amount” is any amount of an agent which, when administered to a subject prone to suffer from a disorder, inhibits the onset of the disorder.

“Preventing” a disease, disorder or condition shall mean stopping the onset or manifestation of the disorder.

“Delaying” a disease, disorder or condition shall mean slowing down or reducing the severity of the manifestation of a disorder.

“Pharmaceutically acceptable carriers” are well known to those skilled in the art and comprise, but are not limited to phosphate buffers or saline. Additionally, such pharmaceutically acceptable carriers can be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers comprise water, alcoholic/aqueous solutions, emulsions and suspensions, including saline and buffered media. Parenteral vehicles comprise sodium chloride solution, Ringer's dextrose, dextrose and sodium chloride, lactated Ringer's and fixed oils. Intravenous vehicles comprise fluid and nutrient replenishers, electrolyte replenishers such as those based on Ringer's dextrose, and the like. Preservatives and other additives may also be present, such as, for example, antimicrobials, antioxidants, chelating agents, inert gases, and the like.

“Administering” means delivering in a manner which is effected or performed using any of the various methods and delivery systems known to those skilled in the art. Administering can be performed, for example, topically, intravenously, pericardially, orally, via implant, transmucosally, transdermally, intramuscularly, subcutaneously, intraperitoneally, intrathecally, intralymphatically, intralesionally, or epidurally. Administering can also be performed, for example, once, a plurality of times, and/or over one or more extended periods.

A “subject” may be any animal, such as a mammal or a bird, including, without limitation, a cow, a horse, a sheep, a pig, a dog, a cat, a rodent such as a mouse or rat, a turkey, a chicken, a primate and a human. The term does not denote a particular age. Thus, adult, newborn and embryonic individuals are intended to be covered.

A “non-human animal” as used herein typically refers to a non-human animal, including, without limitation, farm animals such as cattle, sheep, pigs, goats and horses or domestic mammals such as dogs and cats; laboratory animals including rodents such as mice, rats and guinea pigs; birds, including domestic, wild and game birds such as chickens, turkeys and other gallinaceous birds, ducks, geese, and the like; vertebrates, such as, non-human primates, cows; amphibians; reptiles, etc. The term does not denote a particular age. Thus, both adult, newborn and embryonic individuals are intended to be covered.

A “structural and functional homolog” of a chemical agent is one of a series of structurally and functionally similar agents. A “structural and functional analog” of a chemical agent has a similar structure and function to that of the agent but differs from it in respect to a certain component or components. The term “analog” is broader than and encompasses the term “homolog.” “Analogs” also encompasses the following terms: “isomers” which are chemical compounds that have the same molecular formula but different molecular structures or different spatial arrangement of atoms; “prodrugs” which are functional derivatives of compounds that are readily convertible in vivo into the required compound; and “metabolites” which are the products of biological reactions and comprise active species produced upon introduction of chemical agents into an organism or other biological milieu.

As used herein, the term “nucleic acid molecule” and “polynucleotide” are used interchangeably to refer to a polymeric form of nucleotides of any length, either deoxyribonucleotides or ribonucleotides, or analogs thereof. Polynucleotides may have any three-dimensional structure, and may perform any function, known or unknown. Non limiting examples of polynucleotides comprise a gene, a gene fragment, exons, introns, messenger RNA (mRNA), transfer RNA, ribosomal RNA, ribozymes, cDNA, recombinant polynucleotides, branched polynucleotides plasmids, vectors, isolated DNA of any sequence, isolated RNA of any sequence, nucleic acid probes, and primers. A polynucleotide is typically composed of a specific sequence of four nucleotide bases: adenine (A); cytosine (C); guanine (G); and thymine (T) or uracil (U). Thus, the term polynucleotide sequence is the alphabetical representation of a polynucleotide molecule. This alphabetical representation can be input into databases in a computer having a central processing unit and used for bioinformatics applications such as functional genomics and homology searching.

An “agent” or a “compound” as used herein refers to any compound or substance whose effects (e.g., induction or repression of a specific promoter) can be evaluated using the test animals and methods of the invention. Such compounds comprise, but are not limited to, small organic molecules including pharmaceutically acceptable molecules. Examples of small molecules comprise, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) generally having a molecular weight of less than 10,000 grams per mole salts, esters, and other pharmaceutically acceptable forms of such compounds. Examples of other compounds that can be tested in the methods of the invention comprise polypeptides (e.g., antibodies), peptides, polynucleotides, and polynucleotide analogs, natural products and carbohydrates. Test compounds for use in the methods of the invention can be obtained using any of the numerous approaches in combinatorial methods know in the art including: biological libraries; spatially addressable parallel solid phase or solution phase libraries; synthetic library methods requiring deconvolution; the one-bead one-compound library method; and synthetic library methods using affinity chromatography selection. Many organizations (e.g., the National Institutes of Health, pharmaceutical and chemical corporations) have large libraries of chemical or biological compounds from natural or synthetic processes, or fermentation broths or extracts.

Compounds that Induce ER Stress

ER stress can be induced by various methods known in the art. The use compounds to induce ER-stress or activation of the UPR has been described for tunicamycin (Misra and Pizzo, 2005), thapsigargin (Feng et al., 2003, Nat Cell Biol. 2003 September; 5(9):781-92), free cholesterol (DeVries-Seimon et al., 2005, Cell Biol. 2005 Oct. 10; 171(1):61-73), oxidized cholesterol (Pedruzzi et al., 2004, Mol Cell Biol. 2004 December; 24(24):10703-17), Brefeldin A (Rao et al., J Biol Chem 2001; 276: 33869-33874), homocysteine (Werstuck et al., 2001, J Clin Invest. 107, 1263-1273), Dithiothreitol (Brostrom et al., 1995, J Biol Chem 270, 4127-4132) and A23187 (Dorner et al., 1990, J Biol Chem 265, 22029-22034).

Preparation of Free Cholesterol Induced Macrophages

The FC-induced macrophages can be prepared using methods known in the art, e.g., as described in Yao and Tabas (2000, J. Biol. Chem. 275:23807-23813) and Mori et al. (2001, J. Lipid Res. 42:1771-1781). In one method, macrophages are incubated with acetyl-LDL plus an inhibitor of the cholesterol esterifying enzyme acyl-coenzyme A-cholesterol acyltransferase (ACAT). In anther method, macrophages lacking the ACAT protein are incubated with acetyl-LDL (Wustner et al., Traffic. 2005 May; 6(5):396-412). In a third method, endotoxin-activated macrophages are exposed to atherogenic lipoproteins followed by lipoprotein withdrawal (Funk et al., Atherosclerosis. 1993 Jan. 4; 98(1):67-82).

In some cases, acetyl-low density lipoprotein (acetyl-LDL) and an acyl-coenzyme A:cholesterol acyltransferase (ACAT) inhibitor are used to generate FC-induced apoptotic macrophages. Non-limiting examples of ACAT inhibitors are 58035 (Sandoz Pharmaceutical Corp., East Hanover, N.J.), F 1394 (Fujirebio, Malvern, Pa.), CI-976 (Parke-Davis, Morris Plains, N.J.), and CP-113818 (Pfizer, Inc., Groton, Conn.), or PD-138142-15 (Parke-Davis) are used to induce apoptosis of the macrophages.

The phagocytes used in this type of assay are derived from, for example, peritoneal macrophages that are harvested from an animal by peritoneal lavage. Phagocytes can be identified using methods known in the art, for example using markers such as those described in Cook et al., (2003, J. Immunol. 171(9):4816-4823).

Other methods known in the art for generating a system of phagocytes and apoptotic macrophages can be used for the screens using the general method described supra.

Measuring Vascular Disease

The incidence and progression of vascular disease can be readily measured by methods known in the art.

Quantification of aortic root lesion area can be done by measuring cross-sectional lesion area as described in Teupser et al. (Arterioscler Thromb Vasc Biol. 2003 Oct. 1; 23(10): 1907-13. Epub 2003 Aug. 7). The method can also include the use of hematoxylin and eosin (H&E) staining rather than Oil Red-O staining. Briefly, sections (6-μm thick) are prepared using a microtome from the appearance to the disappearance of the aortic valve leaflets for a total of 56 sections (2 sections/slide for a total of 28 slides). Starting with the first slide, every 5^{th} slide is stained with H&E, and intervening sections are saved for additional analyses. Intimal lesion area (from the lumen to the internal elastic lamina) is quantified using video microscopy and Image-Pro Plus software. Both cellular and acellular areas, including necrotic cores and fibrous caps, are measured. The final lesion area per mouse is calculated as the mean of 5-6 sections. Frozen sections (10-μm thick) are prepared using a cryomicrotome, fixed in 10% neutral-buffered formalin, and stained with H&E. For area analyses a combination of parametric and non-parametric statistical methods can be employed using Statview software (SAS, Inc.). Area datasets can be tested for normality. Because sets of raw lesion data do not usually fit a normal distribution, the data can also be tested following log and square-root transformation. The best-fitting data can be used for parametric analysis (Student's t-test or ANOVA). For area data that do not fit a normal distribution, the non-parametric Mann-Whitney test can be employed.

Measurements of En-face aortic lesion area are described in described in Teupser et al. (Arterioscler Thromb Vasc Biol. 2003 Oct. 1; 23(10): 1907-13. Epub 2003 Aug. 7). In this procedure, the arterial tree is perfused with PBS followed by a fixative containing 4% paraformaldehyde, 5% sucrose, and 20 mM EDTA, pH7.4. The aorta is dissected from the heart through the iliac bifurcation and bathed in PBS at 4° C. The heart and major branches remain attached, and the aorta is opened longitudinally from the aortic root to the iliac arteries. After removal of the heart and major branches, the aorta is mounted onto a black wax surface and imaged with a digital camera. Lesion area is quantified using Image-Pro Plus software and expressed as percent of the total aorta area.

The cellular composition of lesions can be determined by immunohistochemistry using anti-Mac-3 and anti-CD68 for macrophages (BD Phanningen and Serotec), anti-α-actin for SMCs (Zymed Laboratories), anti-factor VIII for endothelial cells (Santa Cruz), and anti-CD3 for T cells (Novocastra, Vector Laboratories). In this approach, sections are first treated with heat and EDTA for antigen retrieval. Endogenous peroxidase activity is blocked with 3% H₂O₂, and non-specific binding is minimized through the use of 10% normal serum. When mouse antibodies are employed, the HistoMouse-SP kit is used to block endogenous IgG and prevent non-specific background. Indirect immunostaining is carried out using the ABComplex/HRP kit (DAKO) followed by diaminobenzidine staining (DAB, Vector Laboratories). Parallel sections using no primary antibody and non-immune isotype-matched antibody serve as a negative controls. Medial staining serves as an internal positive control for the anti-α-actin assay. Spleen tissue serves as a positive control for the CD3 assay. All sections are counter-stained with hematoxylin. The immuno-positive lesion area of stained sections is determined using Image-Pro Plus software and expressed as a percent of the total lesion area.

Quantification of markers of lesion progression can also be used to measure the progression of vascular disease. Necrotic areas may be manifested as acellular areas beneath the fibrous cap or endothelial layer in H&E-stained sections. These areas can be differentiated from regions of dense fibrous scars by the presence of macrophage debris (i.e., immunostaining of macrophage-specific antigens in the absence of cells) in necrotic areas as well as by the absence of collagen staining. Fibrous caps are detected using Verhoeff's stain for elastin (Poly Scientific R&D Corp). The thickness of the cap is then quantified by counting the layers of basement membrane. Collagen-positive areas are detected using Masson's trichrome (Poly Scientific R&D Corp) or Picro-sirius red (Sircol) staining, quantified using Image-Pro Plus software, and expressed as a percent of the total lesion area. All sections are counter-stained with hematoxylin. Medial staining serves as an internal positive control for elastin and collagen.

One skilled in the art will also recognize that an assessment of apoptosis can be used to measure the progression of vascular disease. Apoptosis in lesional cells can be detected by DNA strand breaks and by cleaved (i.e., activated) caspase-3. DNA strand breaks can be quantified by the TUNEL assay (Roche), in which free 3′-OH termini are labeled with terminal deoxynucleotidyl transferase and TMR red nucleotides. Methods can be applied to avoid non-specific labeling due to active RNA synthesis (Kockx et al., Circ Res. 1998 Aug. 24; 83(4):378-87). One section run in parallel without the addition of the transferase enzyme serves as a negative control, and one section treated with DNase serves as a positive control. Activated caspase-3 is detected by an antibody specific for the cleaved form (Cell Signaling Technology). Sections are first treated with heat and EDTA for antigen retrieval, following by IHC using the basic principles, methods, and controls described above. One of the negative controls includes the addition of a blocking caspase-3 peptide (Cell Signaling Technology), and embryonic tissue serves as a positive control. Sections are counter-stained with Hoechst and visualized using video fluoroscopy and RS Image software. The number of cells positive for TUNEL or activated caspase-3 staining are directly counted. Serial sections stained for macrophages, smooth muscle cells, and endothelial cells are used to determine the relative contribution of these cell types to the apoptotic phenotype.

Also readily apparent to one skilled in the art are methods for assess the progression of a vascular disease by assessing of the expression of CHOP and other UPR markers in lesional cells. Immunohistochemistry can be used to detect markers of ER stress and UPR activation. Antibodies can include rabbit anti-phospho-PKR-like ER kinase (PERK, Cell Signaling) as well as polyclonal antibodies specific for glucose-regulated protein (GRP) 78, CHOP, ATF3, and T-cell death-associated gene 51 (TDAG51) (Santa Cruz Biotechnology). Anti-CHOP and anti-TDAG51 are used following heat-induced antigen retrieval, following the basic principles, methods, and controls of IHC. Kidney sections from tunicamycin-injected wild-type or Chop−/− mice serve as positive and negative controls, respectively. All sections are counter-stained with hematoxylin. The immuno-positive areas of stained sections are determined using Image-Pro Plus software and expressed as a percent of the total lesion area. Serial sections stained for M□s, SMCs, ECs, and T cells can be used to determine whether UPR markers colocalize with these cell types. Non-specific staining of aortic root lesions for UPR markers needs to be carefully monitored. Therefore, immunohistochemistry can be complemented with two independent techniques: laser capture microdissection (LCM)/RT-QPCR and XBP-1-venus transgenic mice, (RIKEN) to monitor ER stress by fluorescence. When cells in these mice undergo UPR activation, the XBP-1 transgene is spliced to produce a Venus green fluorescent fusion protein, which can then be visualized by fluorescence microscopy and quantified. The area of fluorescence in atherosclerotic lesions can be quantified and expressed as a percent of the total lesion area. Serial sections can be stained for macrophages SMCs, ECs, and T cells (using red fluorescent secondary antibodies) to determine whether the area of XBP-1-Venus green fluorescence colocalizes with these cell types. For each experiment, aortic root sections from parallel experimental mice without the transgene can be used to distinguish autofluorescence from a true positive signal.

Also readily apparent to one skilled in the art is the use of quantification of gene expression using laser-capture microdissection (LCM)/RT-QPCR in measuring the progression of vascular disease. The method permits an assessment of markers of UPR activation and inflammation in selected cellular regions of plaques (Feg et al., 2003 September; 5(9):781-92. Epub 2003 Aug. 10). In this method, frozen sections (6-μm thick) are cut and mounted on slides and then rapidly immunostained as above to detect macrophages, SMCs, and endothelial cells. Selected regions are melted onto thermoplastic film mounted on optically transparent LCM caps. Microdissected cells are lysed, and RNA is extracted using phenol-chloroform. By way of illustration, CHOP mRNA from the microdissected lysates can be quantified as follows: the RNA is reverse-transcribed into cDNA using oligo-dT and Superscript II (Invitrogen). Quantitative PCR for CHOP and the reference standard cyclophilin A (CypA) is conducted with the Taqman PCR reagent (ABI) and the ABI PRISM 7700 sequence detection system using the forward and reverse primers, probe, and appropriate PCR conditions as determined by one skilled in the art.

Measuring Activation of the UPR Pathway

Methods for assessing activation of the UPR pathway are well characterized in the art. Methods to examine the activation status of the UPR pathway in a cell comprise both immunoassay based methods and methods that involve measuring amounts of cellular RNA. Non-limiting examples of immunoassay based methods to measure UPR activation comprise Western blotting, ELISA, indirect immunofluorescence assays and immunoprecipitation assays. Non-limiting examples of methods involving the measure of an amount of cellular RNA comprise amplification assays (quantitative or semiquantitative PCR) or hybridization assays (Northern blotting, slot blotting, dot blotting, nuclease protection assays or microarray assays).

A measure of UPR activation comprises: an increase in ATF4 expression, an increase in ATF3 expression, an increase in GADD34 expression, an increase in CHOP expression, an increase in XBP1 expression, a dissociation of BiP and PERK, a dissociation of BiP and IRE1, ATF4 nuclear translocation, pATF6(N) nuclear translocation, pAFT6 translocation to the Golgi apparatus, pATF6 proteolytic cleavage, PERK phosphorylation, IRE1 phosphorylation, an increase in the production of ER chaperones, an increase in the production of ER folding enzymes, or any combination thereof. Antibodies to detect activation-induced phosphorylation of UPR pathway proteins or antibodies useful for detecting increases in expression of UPR pathway proteins upon induction of ER stress are well known in the art. Non-limiting examples of commercially available antibodies comprise anti-ATF4 antibodies (Abcam ab23760), anti-ATF3 antibodies (AbNova Corp. H00000467-M04), anti-GADD34 antibodies (IMGENEX, IMG-3001), anti-XBP1 antibodies (Abcom, ab28715), anti-PERK antibodies (Novus Biologicals, H00009451-A01), anti-phospho-PERK antibodies (Cell Signalling Technology, 3191L), anti-BiP antibodies (ABR-Affinity Bioreagents, PA1-014A), anti-IRE1 antibodies (Abcam, ab37073), anti-ATF6 (IMGENEX, IMX-3281) antibodies, anti-CHOP antibodies (Abcam, ab10444), anti-phospho-ERK antibodies (Abcam ab24157), anti-phospho-AKT antibodies (BD Biosciences Pharmingen, 558368, 558316, 558384), anti-XBP-1 antibodies (Abcam, ab37152), anti-phospho-IRS2 (GeneTex, GTX23690) antibodies, and anti-FOXO1 antibodies (Abcam, ab12161).

Monoclonal and polyclonal antibodies useful for measuring UPR pathway activation can also be produced by methods known in the art. Methods to design nucleic acid sequences useful for detecting a specific amount of cellular RNA in a hybridization or amplification based methodology are also known in the art.

A suppression of UPR pathway activation by visfatin (or an agent that increases visfatin activity) can be measured by comparing the increase in the expression of UPR activation induced in a cell in the presence or absence of visfatin treatment (or an agent that increases visfatin activity) under conditions of ER stress. A decrease in the expression of a UPR-induced protein in visfatin treated conditions indicates that visfatin (or an agent that increases visfatin activity) suppresses UPR activation.

Measuring Cell Death

Methods of detecting apoptosis are well known in the art and comprise, for example, cell surface FITC-Annexin V binding assay, DNA laddering assay and TUNEL assay. Assays for apoptosis may be performed by terminal deoxynucleotidyl transferase-mediated digoxigenin-11-dUTP nick end labeling (TUNEL) assay. The TUNEL assay is used to measure nuclear DNA fragmentation characteristic of apoptosis (Lazebnik et al., 1994, Nature 371, 346), by following the incorporation of fluorescein-dUTP (Yonehara et al., 1989, J. Exp. Med. 169, 1747). Apoptosis may further be assayed by acridine orange staining of tissue culture cells (Lucas, R., et al., 1998, Blood 15:4730-41). Other cell-based apoptosis assays comprise the caspase-3/7 assay and the cell death nucleosome ELISA assay. The caspase 3/7 assay is based on the activation of the caspase cleavage activity as part of a cascade of events that occur during programmed cell death in many apoptotic pathways. In the caspase 3/7 assay (commercially available Apo-ONE™ Homogeneous Caspase-3/7 assay from Promega, cat# 67790), lysis buffer and caspase substrate are mixed and added to cells. The caspase substrate becomes fluorescent when cleaved by active caspase 3/7. The nucleosome ELISA assay is a general cell death assay known to those skilled in the art, and available commercially (Roche, Cat# 1774425). This assay is a quantitative sandwich-enzyme-immunoassay which uses monoclonal antibodies directed against DNA and histones respectively, thus specifically determining amount of mono- and oligonucleosomes in the cytoplasmic fraction of cell lysates. Mono and oligonucleosomes are enriched in the cytoplasm during apoptosis due to the fact that DNA fragmentation occurs several hours before the plasma membrane breaks down, allowing for accumulation in the cytoplasm. Nucleosomes are not present in the cytoplasmic fraction of cells that are not undergoing apoptosis. Other methods to investigate the activation of cell death pathways, including, the use of flow cytometry, measuring the levels of phospho-histone H3, phosphorylated MAP kinase, phosphorylated MEK-1, BM28, cyclin E, p53, Rb and PCNA, and the use of apoptosis markers, such as, Annexin V, TUNEL staining, 7-amino-actinomycin D and examining caspase substrate proteolytic cleavage are well described in the art.

Compounds

Compounds useful in the invention comprise, compounds identified using methods described herein. Compounds that can be useful for enhancing visfatin activity associated with advanced atherosclerotic lesions comprise lipoxin, a lipoxin analog (e.g., see U.S. Pat. No. 6,831,186; 15-epi-16-parafluoro-LXA4), or a compound that stimulates lipoxin synthesis or activity such as adenosine 3′5′-cyclic monophosphorothioate, Rp-isomer, triethylammonium salt (Rp-cAMP; Godson et al., 2003, J. Immunol. 164:1663-1667), an apolipoprotein, annexin-I, a biologically active fragment thereof, an annexin-I analog, other compounds used for treatment of autoimmune disorders, a pentarphin such as a cyclopentarphin (see, U.S. patent application publication no. 20050143293), yeast cell wall extract, β1 glucan (see, U.S. Pat. No. 5,786,343), acemannan (see, U.S. Pat. No. 5,106,616), tuftsin (Najjar et al., 1970, Nature 228:672-673); ClqR_{P} ligands (e.g., U.S. Pat. No. 5,965,439), a compound that that can reduce oxidative stress in a cell If such compounds are effective for increasing visfatin activity on macrophages associated with atherosclerotic lesions, they may be modified for targeting to atherosclerotic lesions or delivered using methods that provide them more directly to a lesion. For example, a compound can be delivered to a site identified as containing atherosclerotic lesions using a drug delivery stent. Drug-delivery stents are known in the art (for example, see U.S. Pat. Nos. 6,918,929; 6,758,859; 6,899,729; and 6,904,658), and can be adapted to deliver compounds that enhance visfatin activity, including compounds identified using the methods described herein.

The test compounds of the invention can also be obtained using any of the numerous approaches in combinatorial library methods known in the art, including: biological libraries; peptoid libraries (libraries of molecules having the functionalities of peptides, but with a novel, non-peptide backbone that are resistant to enzymatic degradation but that nevertheless remain bioactive; see, e.g., Zuckermann et al. (1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993, Proc. Natl. Acad. Sci. U.S.A. 90:6909), Erb et al. (1994, Proc. Natl. Acad. Sci. USA 91:11422), Zuckermann et al. (1994, J. Med. Chem. 37:2678) Cho et al. (1993, Science 261:1303), Carrell et al. (1994, Angew. Chem. Int. Ed. Engl. 33:2059), Carell et al. (1994, Angew. Chem. Int. Ed. Engl. 33:2061), and in Gallop et al. (1994, J. Med. Chem. 37:1233).

Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria (Ladner, U.S. Pat. No. 5,223,409), spores (Ladner U.S. Pat. No. 5,223,409), plasmids (Cull et al. (1992, Proc. Natl. Acad. Sci. USA 89:1865-1869) or on phage (Scott and Smith (1990, Science 249:386-390; Devlin, 1990. Science 249:404-406: Cwirla et al., 1990, Proc. Natl. Acad. Sci. 87:6378-6382; Felici, 1991, J. Mol. Biol. 222:301-310; Ladner supra.).

In some cases, a compound that interferes with the activity of a molecule (an inhibitory compound) that inhibits visfatin activity (an inhibitory molecule). An “antisense” nucleic acid can comprise a nucleotide sequence that is complementary to a “sense” nucleic acid encoding a protein, e.g., complementary to the coding strand of a double-stranded cDNA molecule or complementary to an mRNA sequence. The antisense nucleic acid can be complementary to an entire coding strand, or to only a portion thereof. In some cases, the antisense nucleic acid molecule is antisense to a “noncoding region” of the coding strand of a nucleotide sequence (e.g., the 5′ or 3′ untranslated regions).

An antisense nucleic acid can be designed such that it is complementary to the entire coding region of mRNA encoding an inhibitory molecule of visfatin activity, but generally is an oligonucleotide that is antisense to only a portion of the coding or noncoding region of the mRNA. For example, the antisense oligonucleotide can be complementary to the region surrounding the translation start site of the mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid that is useful as described herein can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid can be of an antisense orientation to a target nucleic acid of interest, described further in the following subsection).

The antisense nucleic acid molecules are typically administered to a subject (e.g., by direct injection at a tissue site), or generated in situ such that they hybridize with or bind to cellular mRNA and/or genomic DNA encoding an inhibitory molecule to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are can be used in some embodiments.

In another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al., 1987, Nucleic Acids. Res. 15:6625-6641). The antisense nucleic acid molecule can also comprise a 2′-o-methylribonucleotide (Inoue et al., 1987, Nucleic Acids Res. 15:6131-6148) or a chimeric RNA-DNA analogue (Inoue et al., 1987, FEBS Lett. 215:327-330).

In still another embodiment, an antisense nucleic acid of the invention is a ribozyme. A ribozyme having specificity for an inhibitory molecule encoding nucleic acid can comprise one or more sequences complementary to the nucleotide sequence of the inhibitory molecule and a sequence having known catalytic sequence responsible for mRNA cleavage (see U.S. Pat. No. 5,093,246 or Haselhoff and Gerlach, 1988, Nature 334:585-591). For example, a derivative of a Tetrahymena L-19 IVS RNA can be constructed in which the nucleotide sequence of the active site is complementary to the nucleotide sequence to be cleaved in an inhibitory molecule-encoding mRNA. See, e.g., Cech et al. U.S. Pat. No. 4,987,071; and Cech et al. U.S. Pat. No. 5,116,742. Alternatively, mRNA can be used to select a catalytic RNA having a specific ribonuclease activity from a pool of RNA molecules. See, e.g., Bartel and Szostak, 1993, Science 261:1411-1418.

Gene expression of an inhibitory molecule can be inhibited by targeting nucleotide sequences complementary to the regulatory region of the sequence encoding the molecule (e.g., the promoter and/or enhancers) to form triple helical structures that prevent transcription of the gene in target cells. See generally, Helene, 1991, Anticancer Drug Des. 6:569-84; Helene, 1992, Ann. N.Y. Acad. Sci. 660:27-36; and Maher, 1992, Bioassays 14:807-15. The potential sequences that can be targeted for triple helix formation can be increased by creating a so-called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

A nucleic acid molecule used to inhibit expression of an inhibitory molecule can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup et al., 1996, Bioorganic & Medicinal Chemistry 4: 5-23). As used herein, the terms “peptide nucleic acid” or “PNA” refers to a nucleic acid mimic, e.g., a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. The synthesis of PNA oligomers can be performed using standard solid phase peptide synthesis protocols as described in Hyrup et al., 1996, supra; Perry-O'Keefe et al. 1996, Proc. Natl. Acad. Sci. 93: 14670-14675.

PNAs of nucleic acid molecules corresponding to sequences encoding an inhibitory molecule can be used in therapeutic and diagnostic applications. For example, PNAs can be used as antisense or antigene agents for sequence-specific modulation of gene expression by, for example, inducing transcription or translation arrest or inhibiting replication. PNAs of nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. et al., 1996, supra)); or as probes or primers for DNA sequencing or hybridization (Hyrup B. et al., 1996, supra; Perry-O'Keefe et al., supra).

In other embodiments, the oligonucleotide can comprise other appended groups such as peptides (e.g., for targeting host cell receptors in vivo), or agents facilitating transport across the cell membrane (see, e.g., Letsinger et al., 1989, Proc. Natl. Acad. Sci. USA 86:6553-6556; Lemaitre et al., 1987, Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (see, e.g., Krol et al., 1988, Bio-Techniques 6:958-976) or intercalating agents (see, e.g., Zon, 1988, Pharm. Res. 5:539-549). To this end, the oligonucleotide can be conjugated to another molecule, (e.g., a peptide, hybridization triggered cross-linking agent, transport agent, or hybridization-triggered cleavage agent).

RNA interference (RNAi) is a process whereby double-stranded RNA (dsRNA) induces the sequence-specific degradation of homologous mRNA in animals and plant cells (Hutvagner and Zamore, 2002, Curr. Opin. Genet. Dev. 12:225-232; Sharp, 2001, Genes Dev. 15:485-490). In mammalian cells, RNAi can be triggered by, e.g., approximately 21-nucleotide (nt) duplexes of small interfering RNA (siRNA) (Chiu et al., 2002, Mol. Cell. 10:549-561; Elbashir et al., 2001, Nature 411:494-498), or by micro-RNAs (miRNA), functional small-hairpin RNA (shRNA), or other dsRNAs which are expressed in vivo using DNA templates with RNA polymerase III promoters (Zeng et al., 2002, Mol. Cell. 9:1327-1333; Paddison et al., 2002, Genes Dev., 16:948-958; Lee et al., 2002, Nature Bioteclmol. 20:500-505; Paul et al., 2002, Nature Biotechnol. 20:505-508; Tuschl, 2002, Nature Biotechnol. 20:440-448; Yu et al., 2002, Proc. Natl. Acad. Sci. USA, 99:6047-6052; McManus et al., 2002, RNA 8:842-850; Sui et al., 2002, Proc. Natl. Acad. Sci. USA 99:5515-5520).

Examples of molecules that can be used to decrease expression of an inhibitory molecule comprise double-stranded RNA (dsRNA) molecules that can function as siRNAs targeting nucleic acids encoding the inhibitory molecule and that comprise 16-30, e.g., 16, 17, 18, 19, 20, 21, 22, 23, 24, 25, 26, 27, 28, 29, or 30 nucleotides in each strand, wherein one of the strands is substantially complementary to, e.g., at least 80% (or more, e.g., 85%, 90%, 95%, or 100%) complementary to, e.g., having 3, 2, 1, or 0 mismatched nucleotide(s), a target region, e.g. a transcribed region of a nucleic acid and the other strand is identical or substantially identical to the first strand. The dsRNA molecules can be chemically synthesized, or can be transcribed in vitro from a DNA template, or in vivo from an engineered RNA precursor, e.g., shRNA. The dsRNA molecules may be designed using methods known in the art (e.g., “The siRNA User Guide,” available at rockefeller.edu/labheads/tuschl/siRNA) and can be obtained from commercial sources, e.g., Dharmacon, Inc. (Lafayette, Colo.) and Ambion, Inc. (Austin, Tex.).

Negative control siRNAs generally have the same nucleotide composition as the selected siRNA, but without significant sequence complementarity to the targeted genome. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

The siRNAs for use as described herein can be delivered to a cell by methods known in the art and as described herein in using methods such as transfection utilizing commercially available kits and reagents. Viral infection, e.g., using a lentivirus vector can be used.

An siRNA or other oligonucleotide can also be introduced into the cell by transfection with an heterologous target gene using carrier compositions such as liposomes, which are known in the art, e.g., Lipofectamine™ 2000 (Invitrogen, Carlsbad, Calif.) as described by the manufacturer for adherent cell lines. Transfection of dsRNA oligonucleotides for targeting endogenous genes can be carried out using Oligofectamine™ (Invitrogen, Carlsbad, Calif.). The effectiveness of the oligonucleotide can be assessed by any of a number of assays following introduction of the oligonucleotide into a cell. These assays comprise, but are not limited to, Western blot analysis using antibodies that recognize the targeted gene product following sufficient time for turnover of the endogenous pool after new protein synthesis is repressed, and Northern blot analysis to determine the level of existing target mRNA.

Still further compositions, methods and applications of RNAi technology for use as described herein are provided in U.S. Pat. Nos. 6,278,039, 5,723,750 and 5,244,805, which are incorporated herein by reference.

Conditions Capable of Causing Vascular Disease

One skilled in the art will recognize that there exist a variety of conditions that can increase the likelihood of a subject or a non-human animal having vascular disease. If a method requires that vascular disease be induced in a non-human animal, several such methods are known in the art. These methods comprise several methods to induce thrombotic events, including, direct application of ferric chloride (FeCl3) to the adventitial surface of an artery (Kurz et al., Thromb Res. 1990; 60:269-280), intravenous injection of the photoreactive substance Rose Bengal and the subsequent exposure of an arterial segment to green light (540 nm) (Kikuchi et al. Arterioscler Thromb Vasc Biol. 1998; 18:1069-1078) or a laser pulse, applied through the microscope optics (Falati et al. Nat Med. 2002; 8:1175-1181).

Also known to those skilled in the art are methods to induce a condition of hypercholesterolemia (Pellizzon et al., J Am Coll Nutr. 2007 February; 26(1):66-75) (Hartvigsen et al., Arterioscler Thromb Vasc Biol. 2007 April; 27(4):878-85. Epub 2007 Jan. 25), a condition of hyperlipoproteinemia (Barcat et al., Atherosclerosis. 2006 October; 188(2):347-55. Epub 2005 Dec. 27), a condition of hypertriglyceridemia (Pan et al., Eur J Pharmacol. 2006 May 10; 537(1-3):200-4. Epub 2006 Mar. 10), a condition of lipodystrophy (Shimomura et al., Genes Dev. 1998 Oct. 15; 12(20):3182-94), a condition of hyperglycemia (Botolin et al., J Cell Biochem. 2006 Oct. 1; 99(2):411-24), a condition of reduced HDL (McNeish et al., Proc Natl Acad Sci USA. 2000 Apr. 11; 97(8):4245-50) (Westerterp et al., Arterioscler Thromb Vasc Biol. 2006 November; 26(11):2552-9. Epub 2006 Aug. 31) levels, a condition of elevated LDL levels (Iwaki et al., Blood. 2006 May 15; 107(10):3883-91. Epub 2006 Jan. 24), a condition of low glucose tolerance (Cunha et al., Regul Pept. 2007 Mar. 1; 139(1-3):1-4. Epub 2007 Jan. 4), a condition of insulin resistance (Kim et al., Metabolism. 2007 May; 56(5):676-85), a condition of obesity (Katagiri et al., J Dermatol Sci. 2007 May; 46(2):117-26. Epub 2007 Mar. 9), a condition of dyslipidemia (Mertens et al., Circulation. 2003 Apr. 1; 107(12):1640-6. Epub 2003 Mar. 24), a condition of hyperlipidemia (Graham et al., J Lipid Res. 2007 April; 48(4):763-7. Epub 2007 Jan. 22), a condition of hypercholesterolemia (Cho et al., Exp Mol Med. 2007 Apr. 30; 39(2): 160-9), a condition of vascular restenosis (Schachner et al., Ann Thorac Surg. 2004 May; 77(5):1580-5), a condition of hypertension (Handtrack et al., J Mol Med. 2007 April; 85(4):343-50. Epub 2007 Mar. 2), a condition of Type I diabetes (Botolin and McCabe, Endocrinology. 2007 January; 148(1):198-205. Epub 2006 Oct. 19), a condition of Type II diabetes (Segev et al., Diabetologia. 2007 June; 50(6): 1327-34. Epub 2007 Apr. 19), a condition of hyperinsulinemia (Watson et al., Endocrinology. 2005 December; 146(12):5151-63. Epub 2005 Sep. 1), a condition of atherogenesis (Lewis et al., Circulation. 2007 Apr. 24; 115(16):2178-87. Epub 2007 Apr. 9), a condition of aneurysm (J Vasc Surg. 2006 December; 44(6):1314-21), a condition of ischemia (Fong et al., J Neurosci Res. 2007 May 10; [Epub ahead of print]). Conditions of vascular disease can also be induced by a neointimal hyperplasia following percutaneous a transluminal coronary angiograph (Saito et al., Am J Hematol 1999; 61:238-242), a vascular graft (Fario-Neto et al., Atherosclerosis. 2006 November; 189(1):83-90. Epub 2006 Jan. 18), a coronary artery bypass surgery (Zou et al., Am J Pathol. 1998 October; 153(4):1301-10), a thromboembolic event (Kurz et al., Thromb Res. 1990; 60:269-280), a post-angioplasty restenosis (Pires et al., Heart. 2007 Apr. 20; [Epub ahead of print]), a coronary plaque inflammation (Massberg et al., J Exp Med. 2002 Oct. 7; 196(7):887-96), an embolism (Lockyer et al., Thromb Res. 2006; 118(3):371-80. Epub 2005 Sep. 2), a stroke (Lozano et al., J Neurosci Methods. 2007 May 15; 162(1-2):244-54. Epub 2007 Feb. 1), an arrhythmia (Liu et al., Circ Res. 2006 Aug. 4; 99(3):292-8. Epub 2006 Jul. 6), an atrial fibrillation or atrial flutter (Temple et al., Circ Res. 2005 Jul. 8; 97(1):62-9. Epub 2005 Jun. 9), or a thrombotic occlusion (Liu et al., Thromb Res. 2007 Apr. 26; [Epub ahead of print]) or any combination thereof.

Vascular disease can also be induced in non-human animals by dietary changes. Non-limiting examples of diets known in the art comprise, a high cholesterol diet (Mehta et al., Circ Res. 2007 May 3; [Epub ahead of print]), a high fat diet (Poggi et al., Diabetologia. 2007 June; 50(6):1267-76. Epub 2007 Apr. 11), or a Paigen diet (Paigen et al., Atherosclerosis, 57:65-73 (1985), or a high fat western diet (Kitamoto et al., Circulation. 2007 Apr. 17; 115(15):2065-75. Epub 2007 Apr. 2) or any combination thereof. These methods are provided for illustrative purposes and are not meant to be limiting.

Vascular disease can also be induced in non-human animals by vascular injuries. Several of the methods, including wire injury, electric injury, ligation injury and collar injury are known in the art have been reviewed in Xu Am J Pathol. 2004 July; 165(1):1-10.

Pharmaceutical Compositions

The compounds described herein and identified using methods described herein that are useful for preventing or treating atherosclerosis by enhancing activity of visfatin can be incorporated into pharmaceutical compositions. Such compositions typically comprise the compound and a pharmaceutically acceptable carrier. As used herein the language “pharmaceutically acceptable carrier” comprises solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration comprise parenteral, e.g., intravenous, intradermal, subcutaneous, inhalation, transdermal (topical), transmucosal, and rectal administration; or oral. Solutions or suspensions used for parenteral, intradermal, or subcutaneous application can comprise the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injection use comprise sterile aqueous solutions (where water soluble) or dispersions and sterile powders for the extemporaneous preparation of sterile injectable solutions or dispersion. For intravenous administration, suitable carriers comprise physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluiditv can be maintained, for example, by the use of a coating such as lecithin, by the maintenance of the selected particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In some cases, isotonic agents are included in the composition, for example, sugars, polyalcohols such as manitol, sorbitol, or sodium chloride. Prolonged absorption of an injectable composition can be achieved by including in the composition an agent that delays absorption, for example, aluminum monostearate or gelatin.

Sterile injectable solutions can be prepared by incorporating the active compound in the specified amount in an appropriate solvent with one or a combination of ingredients enumerated above, as needed, followed by filtered sterilization. Generally, dispersions are prepared by incorporating the active compound into a sterile vehicle that contains a basic dispersion medium and other ingredients selected from those enumerated above or others known in the art. In the case of sterile powders for the preparation of sterile injectable solutions, the methods of preparation comprise vacuum drying and freeze-drying which yields a powder of the active ingredient plus any additional desired ingredient from a previously sterile-filtered solution thereof.

Oral compositions generally comprise an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be comprised as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and comprise, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the selected pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices can be used in some embodiments. While compounds that exhibit toxic side effects may be used, it is generally desirable to design a delivery system that targets such compounds to the focal site of the disease, e.g., atherosclerotic lesions, to minimize potential damage to unaffected cells are tissues, thereby reducing side effects.

The data obtained from the cell culture assays and animal studies can be used in formulating a range of dosage for use in humans. The dosage of such compounds generally lies within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

As defined herein, a therapeutically effective amount of protein or polypeptide (i.e., an effective dosage) ranges from about 0.001 to 30 mg/kg body weight, about 0.01 to 25 mg/kg body weight, about 0.1 to 20 mg/kg body weight, about 1 to 10 mg/kg, about 2 to 9 mg/kg, about 3 to 8 mg/kg, about 4 to 7 mg/kg, or about 5 to 6 mg/kg body weight. The protein or polypeptide can be administered one time per week for between about 1 to 10 weeks, for example, between 2 to 8 weeks, between about 3 to 7 weeks, about 4, 5, or 6 weeks, or chronically. The skilled artisan will appreciate that certain factors may influence the dosage and timing to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can comprise a single treatment or can comprise a series of treatments.

For antibodies, the dosage is generally 0.1 mg/kg of body weight (for example, 10 mg/kg to 20 mg/kg). If the antibody is to act in the brain, a dosage of about 50 mg/kg to 100 mg/kg is usually appropriate. Generally, partially human antibodies and fully human antibodies have a longer half-life within the human body than other antibodies. Accordingly, lower dosages and less frequent administration are possible. Modifications such as lipidation can be used to stabilize antibodies and to enhance uptake and tissue penetration (e.g., into the brain). A method for lipidation of antibodies is described in Cruikshank et al. (1997, J. Acquired Immune Deficiency Syndromes and Human Retrovirology 14:193).

In general, a compound that can enhance visfatin activity associated with advanced atherosclerotic lesions is administered to a high-risk subject in an acute or semi-acute setting to stabilize their plaques (lesions). The subject can then be maintained on the compound for a sufficient time to allow the plaque-stabilizing effects of a simultaneously administered cholesterol-lowering drug to become manifest, for example, for about one to two years or longer.

The invention encompasses compounds that modulate visfatin effects on phagocytes associated with advanced atherosclerotic lesions. A compound can, for example, be a small molecule. For example, such small molecules comprise, but are not limited to, peptides, peptidomimetics (e.g., peptoids), amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram perkilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The compounds described herein can be conjugated to another moiety such as an antibody, for example, for targeting the compound for delivery to advanced atherosclerotic lesions.

Nucleic acid molecules that are identified for use as compounds useful for enhancing visfatin activity as described herein can be inserted into vectors and used as gene therapy vectors. Gene therapy vectors can be delivered to a subject by, for example, intravenous injection, local administration (see, U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can comprise the gene therapy vector in an acceptable diluent, or can comprise a slow release matrix in which the gene delivery vehicle is embedded. Alternatively, where the complete gene delivery vector can be produced intact from recombinant cells, e.g., retroviral vectors, the pharmaceutical preparation can comprise one or more cells which produce the gene delivery system. Other methods of delivery of nucleic acids as gene therapy vectors that are known in the art can also be used. Such methods can be combined with other targeted delivery methods such as a stent. Methods of constructing and using drug-delivery stents are known in the art, and some are cited supra.

The pharmaceutical compositions can be included in a container, pack, or dispenser together with instructions for administration.

Methods of Treatment

Provided herein are both prophylactic and therapeutic methods of treating a subject at risk of (or susceptible to) having atherosclerosis, in particular, advanced atherosclerosis, characterized by having advanced atherosclerotic lesions. As used herein, the term “treating” is defined as the application or administration of a therapeutic agent to a subject (e.g., a non-human animal or a human) in need thereof with the purpose to cure, heal, alleviate, relieve, alter, remedy, ameliorate, improve or affect the disease, the symptoms of disease or the predisposition toward disease. Subjects comprise, for example, individuals having at least one of a history of heart disease, diabetes, arteriosclerosis, hypercholesterolemia, hypertension, cigarette smoking, obesity, metabolic syndrome, physical inactivity or other disorders or symptoms associated with atherosclerosis (e.g., see The Merck Manual, Sixteenth Edition, Berkow, ed., Merck Research Laboratories, Rahway, N.J., 1992). A therapeutic agent comprises, but is not limited to, small molecules, peptides, antibodies, ribozymes, antisense oligonucleotides, siRNA and other compounds described herein.

In one aspect, the invention provides a method for preventing in a subject, a disease or condition associated with phagocyte cell death associated with advanced atherosclerotic lesions by administering to the subject a compound that enhances the survival of phagocytes associated with advanced atherosclerotic lesions. The compound can suppress cholesterol overload-induced UPR pathway activation in cells associated with advanced atherosclerotic lesions, cell death of phagocytes associated with advanced atherosclerotic lesions, or both.

Subjects at risk for having advanced atherosclerotic lesions can be identified by methods known in the art, which can comprise angiography, ultrasound, CT scan, or other indicia of atherosclerosis. In addition, symptoms of atherosclerosis such as critical stenosis, thrombosis, aneurysm, embolus, decreased blood flow to a tissue, angina on exertion, bruit can be used to identify a subject having or at risk for atherosclerosis. Administration of a prophylactic agent can occur prior to the manifestation of symptoms characteristic of having atherosclerosis or advanced atherosclerotic lesions such that disease or disorder is prevented or, alternatively, delayed in its progression.

As discussed herein, compounds, e.g., an agent identified using an assay described above, that exhibits the enhance the ability of visfatin to suppress UPR pathway activation-induced cell death, particularly phagocyte death associated with advanced atherosclerotic lesions, can be used in accordance with prevention or treatment methods described herein to prevent and/or ameliorate symptoms of atherosclerosis. Such molecules can comprise, but are not limited to peptides, phosphopeptides, peptoids, small non-nucleic acid organic molecules, inorganic molecules, and proteins including, for example, antibodies (e.g., polyclonal, monoclonal, humanized, anti-idiotypic, chimeric or single chain antibodies, and Fab, F(ab′)₂ and Fab expression library fragments, scFV molecules, and epitope-binding fragments thereof).

Further, oligonucleotides including antisense, siRNA and ribozyme molecules that inhibit expression of a gene whose product inhibits visfatin activity can also be used in accordance with the invention to increase the level of visfatin activity. Still further, triple helix molecules can be utilized in reducing the level of activity of such a gene product. Antisense, ribozyme and triple helix molecules are discussed above. In some cases, compounds that increase the expression, and thereby the activity of a gene product that is associated with increased visfatin activity are used in a method for preventing or treating atherosclerosis. In such cases, nucleic acid molecules that encode and express such gene products (polypeptides) are introduced into cells via gene therapy methods. In some cases, precursor cells for phagocytes (e.g., monocytes) are obtained, in general from the subject to be treated, and the precursor cells are subjected ex vivo to gene therapy to introduce the desired nucleic acid sequence encoding a polypeptide or a regulatory nucleic acid sequence that is introduced into the genome of the phagocyte precursor cell in such a way that it promotes expression of an endogenous gene that increases visfatin activity. The precursor cell is then introduced into the subject as a treatment method.

Another method by which nucleic acid molecules are utilized in treating or preventing atherosclerosis is through the use of aptamer molecules specific for a protein that, when contacted by a binding partner, promotes visfatin activity, e.g., in advanced atherosclerotic lesions. Aptamers are nucleic acid molecules having a tertiary structure which permits them to specifically bind to protein ligands (see, e.g., Osborne, et al., 1997, Curr. Opin. Chem. Biol. 1:5-9; and Patel, 1997, Curr. Opin. Chem. Biol. 1:32-46). Since nucleic acid molecules may in many cases be more conveniently introduced into target cells than therapeutic protein molecules may be, aptamers offer a method by which visfatin activity can be specifically enhanced without the introduction of drugs or other molecules that may have pluripotent effects.

Antibodies or biologically active fragments thereof that are useful as compounds for enhancing visfatin activity associated with atherosclerosis can be generated and identified using methods known in the art. Such antibodies or fragments can be administered to a subject enhance visfatin activity to treat or prevent atherosclerosis.

In instances where the target antigen is intracellular and whole antibodies are used, internalizing antibodies can be used. Lipofectin™ or liposomes can be used to deliver the antibody or a fragment of the Fab region that binds to the target antigen into cells. Where fragments of the antibody are used, the smallest inhibitory fragment that binds to the target antigen is generally used. For example, peptides having an amino acid sequence corresponding to the Fv region of the antibody can be used. Alternatively, single chain neutralizing antibodies that bind to intracellular target antigens can also be administered. Such single chain antibodies can be administered, for example, by expressing nucleotide sequences encoding single-chain antibodies within the target cell population (see e.g., Marasco et al. (1993, Proc. Natl. Acad. Sci. USA 90:7889-7893).

The identified compounds that increase visfatin activity as described herein can be administered to a subject at therapeutically effective doses to prevent, treat or ameliorate atherosclerosis. A therapeutically effective dose refers to that amount of the compound sufficient to result in amelioration of at least one symptom of the disorder. Toxicity and therapeutic efficacy of such compounds can be determined by standard pharmaceutical procedures known in the art.

Data obtained from the cell culture assays and animal studies can be used in formulating a range of dosage for use in humans. The dosage of such compounds generally lies within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Another example of determination of effective dose for an individual is the ability to directly assay levels of “free” and “bound” compound in the serum of the test subject. Such assays may utilize antibody mimics and/or “biosensors” that have been created through molecular imprinting techniques. The compound which is able to increase phagocyte survival associated with advanced atherosclerotic lesions is used as a template, or “imprinting molecule”, to spatially organize polymerizable monomers prior to their polymerization with catalytic reagents. The subsequent removal of the imprinted molecule leaves a polymer matrix that contains a repeated “negative image” of the compound and is able to selectively rebind the molecule under biological assay conditions. A detailed review of this technique can be seen in Ansell et al. (1996, Curr. Opin. Biotechnol. 7:89-94) and in Shea. (1994, Trends Polymer Sci. 2:166-173. Such “imprinted” affinity matrixes are amenable to ligand-binding assays, whereby the immobilized monoclonal antibody component is replaced by an appropriately imprinted matrix. An example of the use of such matrixes in this way can be seen in Vlatakis et al. (1993, Nature 361:645-647). Through the use of isotope-labeling, the “free” concentration of compound that increases visfatin activity can be monitored and used in calculations of IC₅₀.

Pharmaceutical Formulations and Modes of Administration

In one aspect, a pharmaceutical of the invention comprises a substantially purified protein, nucleic acid, or chemical (e.g., substantially free from substances that limit its effect or produce undesired side-effects). The subject can be an animal, including but not limited to animals such as cows, pigs, horses, chickens, cats, dogs, etc., and can be a mammal and a human.

Various delivery systems are known and can be used to administer the pharmaceutical of the invention, e.g., encapsulation in liposomes, microparticles, microcapsules, receptor-mediated endocytosis (see, e.g., Wu and Wu, 1987, J. Biol. Chem. 262:4429-4432), construction of a nucleic acid as part of a retroviral or other vector, etc. Methods of introduction comprise but are not limited to intradermal, intramuscular, intraperitoneal, intravenous, subcutaneous, intranasal, epidural, and oral routes. Nucleic acids and proteins of the invention may be administered by any convenient route, for example by infusion or bolus injection, by absorption through epithelial or mucocutaneous linings (e.g., oral mucosa, rectal and intestinal mucosa, etc.) and may be administered together with other biologically active agents such as chemotherapeutic agents. Administration can be systemic or local.

In a specific embodiment, it may be desirable to administer the nucleic acid or protein of the invention by injection, by means of a catheter, by means of a suppository, or by means of an implant, said implant being of a porous, non-porous, or gelatinous material, including a membrane, such as a sialastic membrane, or a fiber. When administering a protein, including an antibody, of the invention, care must be taken to use materials to which the protein does not absorb.

In another embodiment, the compound or composition can be delivered in a vesicle, in particular a liposome (see Langer, 1990, Science 249:1527-1533; Treat et al., 1989, in Liposomes in the Therapy of Infectious Disease and Cancer, Lopez-Berestein and Fidler (eds.), Liss, New York, pp. 353-365; Lopez-Berestein, ibid., pp. 317-327; see generally, ibid.).

In yet another embodiment, the compound or composition can be delivered in a controlled release system. In one embodiment, a pump may be used (see Langer, supra; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used (see Medical Applications of Controlled Release, 1974, Langer and Wise (eds.), CRC Pres., Boca Raton, Fla.; Controlled Drug Bioavailability, Drug Product Design and Performance, 1984, Smolen and Ball (eds.), Wiley, New York; Ranger and Peppas, 1983, Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105).

Other controlled release systems are discussed in the review by Langer, 1990, Science 249:1527-1533.

In a specific embodiment where a nucleic acid of the invention is administered, the nucleic acid can be administered in vivo to promote expression of its encoded protein or RNA molecule, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. 26. USA 88:1864-1868), etc. Alternatively, a nucleic acid can be introduced intracellularly and incorporated within host cell DNA for expression, by homologous recombination.

The invention also provides pharmaceutical compositions (pharmaceuticals of the invention). Such compositions comprise a therapeutically effective amount of a nucleic acid, chemical or protein of the invention, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water can be a carrier when the pharmaceutical composition is administered intravenously. Saline solutions and aqueous dextrose and glycerol solutions can also be employed as liquid carriers, particularly for injectable solutions. Suitable pharmaceutical excipients comprise starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can comprise standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the nucleic acid or protein of the invention, and can be in purified form, together with a suitable amount of carrier so as to provide the form for proper administration to the patient. The formulation should suit the mode of administration.

In another embodiment, the pharmaceutical of the invention is formulated in accordance with routine procedures as a pharmaceutical composition adapted for intravenous administration to human beings. Typically, compositions for intravenous administration are solutions in sterile isotonic aqueous buffer. Where necessary, the pharmaceutical of the invention may also comprise a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical of the invention is to be administered by infusion, it can be dispensed with an infusion bottle containing sterile pharmaceutical grade water or saline. Where the pharmaceutical of the invention is administered by injection, an ampoule of sterile water for injection or saline can be provided so that the ingredients may be mixed prior to administration.

For buccal administration the compositions may take the form of tablets or lozenges formulated in conventional manner.

For administration by inhalation, the compounds for use according to the invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The amount of the nucleic acid or protein of the invention which will be effective in the treatment or prevention of the indicated disease can be determined by standard clinical techniques. In addition, in vitro assays may optionally be employed to help identify optimal dosage ranges. The precise dose to be employed in the formulation will also depend on the route of administration, and the stage of indicated disease, and should be decided according to the judgment of the practitioner and each patient's circumstances. Effective doses may be extrapolated from dose-response curves derived from in vitro or animal model test systems.

Protein Purification

Generally, the protein of the invention can be recovered and purified from recombinant cell cultures by known methods, including ammonium sulfate precipitation, acid extraction, anion or cation exchange chromatography, phosphocellulose chromatography, immunoaffinity chromatography, hydroxyapatite chromatography, and lectin chromatography. Before the protein of the invention can be purified, total protein has to be prepared from the cell culture. This procedure comprises collection, washing and lysis of said cells and is well known to the skilled artisan.

However, the invention provides methods for purification of the protein of the invention which are based on the properties of the peptide tag present on the protein of the invention. One approach is based on specific molecular interactions between a tag and its binding partner. The other approach relies on the immunospecific binding of an antibody to an epitope present on the tag or on the protein which is to be purified. The principle of affinity chromatography well known in the art is generally applicable to both of these approaches.

Described below are several methods based on specific molecular interactions of a tag and its binding partner.

A method that is generally applicable to purifying protein of the invention that are fused to the constant regions of immunoglobulin is protein A affinity chromatography, a technique that is well known in the art. Staphylococcus protein A is a 42 kD polypeptide that binds specifically to a region located between the second and third constant regions of heavy chain immunoglobulins. Because of the Fc domains of different classes, subclasses and species of immunoglobulins, affinity of protein A for human Fc regions is strong, but may vary with other species. Other subclasses comprise human IgG-3, and most rat subclasses. For certain subclasses, protein G (of Streptococci) may be used in place of protein A in the purification. Protein-A sepharose (Pharmacia or Biorad) is a commonly used solid phase for affinity purification of antibodies, and can be used essentially in the same manner for the purification of the protein of the invention fused to an immunoglobulin Fc fragment. Bound protein of the invention can be eluted by various buffer systems known in the art, including a succession of citrate, acetate and glycine-HCL buffers which gradually lowers the pH. The recombinant cells can also produce antibodies which will be copurified with the protein of the invention. See, for example, Langone, 1982, J. Immunol. meth. 51:3; Wilchek et al., 1982, Biochem. Intl. 4:629; Sjobring et al., 1991, J. Biol. Chem. 26:399; page 617-618, in Antibodies A Laboratory Manual, edited by Harlow and Lane, Cold Spring Harbor laboratory, 1988.

Alternatively, a polyhistidine tag may be used, in which ease, the protein of the invention can be purified by metal chelate chromatography. The polyhistidine tag, usually a sequence of six histidines, has a high affinity for divalent metal ions, such as nickel ions (Ni.sup.2+), which can be immobilized on a solid phase, such as nitrilotriacetic acid-matrices. Polyhistidine has a well characterized affinity for Ni.sup.2+-NTA-agarose, and can be eluted with either of two mild treatments:imidazole (0.1-0.2 M) will effectively compete with the resin for binding sites; or lowering the pH just below 6.0 will protonate the histidine sidechains and disrupt the binding. The purification method comprises loading the cell culture lysate onto the Ni.sup.2+-NTA-agarose column, washing the contaminants through, and eluting the protein of the invention with imidazole or weak acid. Ni.sup.2+-NTA-agarose can be obtained from commercial suppliers such as Sigma (St. Louis) and Qiagen. Antibodies that recognize the polyhistidine tag are also available which can be used to detect and quantitate the protein of the invention.

Another exemplary peptide tag that can be used is the glutathione-S-transferase (GST) sequence, originally cloned from the helminth, Schistosoma japonicum. In general, a protein of the invention-GST fusion expressed in a prokaryotic host cell, such as E. coli, can be purified from the cell culture lysate by absorption with glutathione agarose beads, followed by elution in the presence of free reduced glutathione at neutral pH. Since GST is known to form dimers under certain conditions, dimeric protein of the invention may be obtained. See, Smith, 1993, Methods Mol. Cell Bio. 4:220-229.

Another useful peptide tag that can be used is the maltose binding protein (MEP) of E. coli, which is encoded by the malE gene. The protein of the invention binds to amylose resin while contaminants are washed away. The bound protein of the invention-MBP fusion is eluted from the amylose resin by maltose. See, for example, Guan et al., 1987, Gene 67:21-30.

The second approach for purifying the protein of the invention is applicable to peptide tags that contain an epitope for which polyclonal or monoclonal antibodies are available. It is also applicable if polyclonal or monoclonal antibodies specific to the protein of the invention are available. Various methods known in the art for purification of protein by immunospecific binding, such as immunoaffinity chromatography, and immunoprecipitation, can be used. See, for example, Chapter 13 in Antibodies A Laboratory Manual, edited by Harlow and Lane, Cold Spring Harbor laboratory, 1988; and Chapter 8, Sections I and II, in Current Protocols in Immunology, ed. by Coligan et al., John Wiley, 1991; the disclosure of which are both incorporated by reference herein.

Gene Therapy Approaches

In a specific embodiment, nucleotide sequences encoding, visfatin, a visfatin polypeptide, a polypeptide that increases visfatin activity or nucleotide sequences encoding therapeutic RNA molecules, such as antisense RNA are administered to treat, or prevent-various diseases. These nucleotide sequences are collectively referred to as nucleotide sequences of the invention. Gene therapy refers to therapy performed by the administration to a subject of an expressed or expressible nucleotide sequence. In this embodiment of the invention, the nucleotide sequences produce their encoded protein or RNA molecule that mediates a therapeutic effect.

Any of the methods for gene therapy available in the art can be used according to the invention. Exemplary methods are described below.

For general reviews of the method of gene therapy, see, Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; Morgan and Anderson, 1993, Ann. Rev. Biochem. 62:191-217; May, 1993, TIBTECH 1, 1(5):155-215. Methods commonly known in the art of recombinant DNA technology which cap be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a specific embodiment, nucleic acid molecules are used in which the nucleotide sequence of the invention is flanked by regions that promote homologous recombination at a desired site in the genome, thus providing for intrachromosomal expression of the nucleotide sequence of the invention (Koller and Smithies, 1989, Proc. Natl. Acad. Sci. USA 86:8932-8935; Zijistra et al., 1989, Nature 342:435-438).

In a specific embodiment, the nucleic acid sequences are directly administered in vivo, where it is expressed to produce the encoded product. This can be accomplished by any of numerous methods known in the art, for example by constructing them as part of an appropriate nucleic acid expression vector and administering the vector so that the nucleic acid sequences become intracellular. Gene therapy vectors can be administered by infection using defective or attenuated retrovirals or other viral vectors (see, e.g., U.S. Pat. No. 4,980,286); direct injection of naked DNA; use of microparticle bombardment (e.g., a gene gun; Biolistic, Dupont); coating with lipids or cell-surface receptors or transfecting agents; encapsulation in liposomes, microparticles, or microcapsules; administration in linkage to a peptide which is known to enter the nucleus; administration in linkage to a ligand subject to receptor-mediated endocytosis (see, e.g., Wu and Wu, 1987, J. Biol. Chem. 262:4429-4432) (which can be used to target cell types specifically expressing the receptors); etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g. PCT Publications WO 92/06 180; WO 92/22635: WO92/20316; WO93/14188, and WO 93/20221). Alternatively, the nucleic acid can be introduced intracellularly and-incorporated within host cell DNA for expression by homologous recombination (Koller and Smithies, 1989, Proc.-Ni.tl. Acad. Sci. USA 86:8932-8935; Zijlstra et al., 1989, Nature 342:435-438).

In a specific embodiment, viral vectors that contain the nucleotide sequence of the invention are used. For example, a retroviral vector can be used (see Miller et al., 1993, Meth. Enzymol. 217:581-599). These retroviral vectors contain the components necessary for the correct packaging of the viral genome and integration into the host cell DNA. The nucleotide sequences of the invention to be used in gene therapy are cloned into one or more vectors, thereby facilitating delivery of the gene into a patient. More detail about retroviral vectors can be found in Boesen et al., 1994; Biotherapy 6:29 1-302, which describes the use of a retroviral vector to deliver the mdr 1 gene to hematopoietic stem cells in order to make the stem cells more resistant to chemotherapy. Other references illustrating the use of retroviral vectors in gene therapy are: Clowes et al., 1994, J. Clip. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

The following animal regulatory regions, which exhibit tissue specificity and have been utilized in transgenic animals, can be used for expression in a particular tissue or cell type: scavenger receptor gene control region which is active in macrophages (Fan et al., 2004, Transgenic Research Volume 13:261-269); elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in the liver (Pinkert et al., 1987, Genes and Devel. 1:268-276), alpha-fetoprotein gene control region which is active in the liver (Krumlauf et al., 1985, Mol. Cell. Biol. 5:1639-1648: Hammer et al., 1987, Science 235:53-58; alpha 1-antitrypsin gene control region which is active in the liver (Kelsey et al., 1987, Genes and Devel. 1:161-171), beta-globin gene control region which is active in myeloid cells (Mograrn et al., 1985, Nature 315:338-340; Kollias et al., 1986, Cell 46:89-94; myelin basic protein gene control region which is active in oligodendrocyte cells in the brain (Readhead et al., 1987, Cell 48:703-712), myosin light chain-2 gene control region which is active in skeletal muscle (Sani, 1985, Nature 314:283-286), tyrosine hydroxylase (TH) gene control region which is active in catecholaminergic neurons (Klejbor et al., J Neurochem. 2006 June; 97(5):1243-58. Epub 2006 Mar. 8), dopamine beta-hydroxylase gene control region which is active in sympathetic and other neurons (Mercer et al., Neuron. 1991 November; 7(5):703-16) and gonadotropic releasing hormone gene control region which is active in the hypothalamus (Mason et al., 1986, Science 234:1372-1378).

Inhibitory Antisense, Ribozyme and Triple Helix Molecules

Among the compounds that may exhibit the ability to modulate the activity of visfatin are antisense, ribozyme, and triple helix molecules. Techniques for the production and use of such molecules are well known to those of skill in the art. For example, antisense targeting visfatin mRNA inhibits visfatin signaling, as described in Section & (see FIGS. 12 and 13).

Antisense RNA and DNA molecules act to directly block the translation of mRNA by hybridizing to targeted mRNA and preventing protein translation. Antisense approaches involve the design of oligonucleotides that are complementary to a target gene mRNA. The antisense oligonucleotides will bind to the complementary target gene mRNA transcripts and prevent translation. Absolute complementarity, is not required.

A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Nucleic acid hybridization is a fundamental physiochemical process, central to the understanding of molecular biology. Probe-based assays use hybridization for the detection, quantitation and analysis of nucleic acids. Nucleic acid probes have been used to analyze samples from a variety of sources for the presence of nucleic acids, as well as to examine clinical conditions of interest in single cells and tissues.

Fundamental to the understanding of nucleic acid probes is the understanding of hybrid melting temperatures (Tm). The Tm of a probe-target hybrid is an idealized equilibrium, defined as the temperature at which 50% of the probes are hybridized, and 50% are non-hybridized. This equilibrium is dependant on several factors including salt concentration, probe concentration, target concentration, and pH.

Generally, hybridization assays are designed to achieve high specificity, meaning that probes only hybridize to perfectly matched (fully complementary), or nearly perfectly matched (partially complementary) targets. Many technologies have been developed to aid in achieving high specificity. For example, denaturants such as formamide, urea, or formaldehyde can be used to lower the effective Tm of a probe. Chaotropic salts such as guanidinium thiocyanate, tetramethylammonium chloride, guanidinium hydrochloride, sodium thiocyanate and others used at high concentrations disrupt the formation of hydrogen bonds. Manipulations of the factors defined by Tm such as temperature, or probe concentration directly affect the specificity of probes. Other factors such as competition with other probes, or use of blocker probes (see U.S. Pat. No. 6,110,676) will also affect the specificity.

In one embodiment, oligonucleotides complementary to non-coding regions of the JNK2 gene could be used in an antisense approach to inhibit translation of endogenous JNK2 mRNA. Antisense nucleic acids should be at least six nucleotides in length, and can be oligonucleotides ranging from 6 to about 50 nucleotides in length. In specific aspects the oligonucleotide is at least 10 nucleotides, at least 17 nucleotides, at least 25 nucleotides or at least 50 nucleotides.

Regardless of the choice of target sequence, in vitro studies are can first be performed to quantitate the ability of the antisense oligonucleotide to inhibit gene expression. These studies can utilize controls that distinguish between antisense gene inhibition and nonspecific biological effects of oligonucleotides. These studies can also compare levels of the target RNA or protein with that of an internal control RNA or protein. Additionally, it is envisioned that results obtained using the antisense oligonucleotide are compared with those obtained using a control oligonucleotide. In some embodiments, the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may comprise other appended groups such as peptides (e.g., for targeting host cell receptors in vivo), or agents facilitating transport across the cell membrane (see, e.g., Letsinger, et al., 1989, Proc. Natl. Acad. Sci. U.S.A. 86, 6553-6556; Lemaitre, et al., 1987, Proc. Natl. Acad. Sci. 84, 648-652; PCT Publication No. WO88/09810, published Dec. 15, 1988) or the blood-brain barrier (see, e.g., PCT Publication No. WO89/10134, published Apr. 25, 1988), hybridization-triggered cleavage agents (see, e.g., Krol et al., 1988, Bio Techniques 6, 958-976) or intercalating agents (see, e.g., Zon, 1988, Pharm. Res. 5, 539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxamthine, xanthine, 4-acetylcytosine, 5-(carboxyhydroxylmethyl)uracil, 5-carboxymethylaminomethyl-w-thiouridine, 5-carboxymethylaminomethyluracil, dhiydrouracil, beta-D-galactosylqueosine, inosine, N6-isopentenyladenine, 1-methylguanine, 1-methylinosine, 2,2-dimethylguanine, 2-methyladenine, 5-methylaminomethyluracil, 5-methoxyaminomethyl-w-thiouracil, beta-D-mannsylqueosine, 5-methoxycarboxymetholuracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thioracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate (S-ODNs), a phosphorodithioate, a phosphoramidothioate, a phosphoramidate, a phosphordiamidate, a methylphosphonate, an alkyl phosphotriester, and a formxacetal or analog thereof.

In yet another embodiment, the antisense oligonucleotide is an-anomeric oligonucleotide. An-anomeric oligonucleotide forms specific double-stranded hybrids with complementary RNA in which, contrary to the usual-units, the strands run parallel to each other (Gautier, et al., 1987, Nucl. Acids Res. 15, 6625-6641). The oligonucleotide is a 2-O-methylribonucleotide (Inoue, et al., 1987, Nucl. Acids Res. 15, 6131-6148), or a chimeric RNA-DNA analogue (Inoue, et al., 1987, FEBS Lett. 215, 327-330).

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein, et al. (1988, Nucl. Acids Res. 16, 3209), methylphosphonate oligonucleotides can be prepared by use of controlled pore glass polymer supports (Sarin, et al., 1988, Proc. Natl. Acad. Sci. U.S.A. 85, 7448-7451), etc.

While antisense nucleotides complementary to the target gene coding region sequence could be used, those complementary to the transcribed, untranslated region are contemplated.

In one embodiment of the invention, gene expression downregulation is achieved because specific target mRNAs are digested by RNAse H after they have hybridized with the antisense phosphorothioate oligonucleotides (S-ODNs). Since no rules exist to predict which antisense S-ODNs will be more successful, the best strategy is completely empirical and consists of trying several antisense S-ODNs. Antisense phosphorothioate oligonucleotides (S-ODNs) will be designed to target specific regions of mRNAs of interest. Control S-ODNs consisting of scrambled sequences of the antisense SODNs will also be designed to assure identical nucleotide content and minimize differences potentially attributable to nucleic acid content. All S-ODNs will be synthesized by Oligos Etc. (Wilsonville, Oreg.). In order to test the effectiveness of the antisense molecules when applied to cells in culture, such as assays for research purposes or ex vivo gene therapy protocols, cells will be grown to 60-80% confluence on 100 mm tissue culture plates, rinsed with PBS and overlaid with lipofection mix consisting of 8 ml Opti-MEM, 52.8 l Lipofectin, and a final concentration of 200 nM S-ODNs. Lipofections will be carried out using Lipofectin Reagent and Opti-MEM (Gibco BRL). Cells will be incubated in the presence of the lipofection mix for 5 hours. Following incubation the medium will be replaced with complete DMEM. Cells will be harvested at different time points postlipofection and protein levels will be analyzed by Western blot.

Antisense molecules should be targeted to cells that express the target gene, either directly to the subject in vivo or to cells in culture, such as in ex vivo gene therapy protocols. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

However, it is often difficult to achieve intracellular concentrations of the antisense sufficient to suppress translation of endogenous mRNAs. Therefore one approach utilizes a recombinant DNA construct in which the antisense oligonucleotide is placed under the control of a strong pol III or pol H promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced e.g. such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian cells. Such promoters can be inducible or constitutive. Such promoters include but are not limited to: the SV40 early promoter region (Bernoist and Chambon, 1981, Nature 290, 304-310), the promoter contained in the 3 long terminal repeat of Rous sarcoma virus (Yamamoto, et al., 1980, Cell 22, 787-797), the herpes thymidine kinase promoter (Wagner, et al., 1981, Proc. Natl. Acad. Sci. U.S.A. 78, 1441-1445), the regulatory sequences of the metallothionein gene (Brinster, et al., 1982, Nature 296, 39-42), etc. Any type of plasmid, cosmid, YAC or viral vector can be used to prepare the recombinant DNA construct which can be introduced directly into the tissue site. Alternatively, viral vectors can be used that selectively infect the desired tissue, in which case administration may be accomplished by another route (e.g., systemically).

Ribozyme molecules designed to catalytically cleave target gene mRNA transcripts can also be used to prevent translation of target gene mRNA and, therefore, expression of target gene product (see, e.g., PCT International Publication WO90/11364, published Oct. 4, 1990; Sarver, et al., 1990, Science 247, 1222-1225).

Ribozymes are enzymatic RNA molecules capable of catalyzing the specific cleavage of RNA. (For a review, see Rossi, 1994, Current Biology 4, 469-471). The mechanism of ribozyme action involves sequence specific hybridization of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage. For this sequence, see, e.g., U.S. Pat. No. 5,093,246, which is incorporated herein by reference in its entirety.

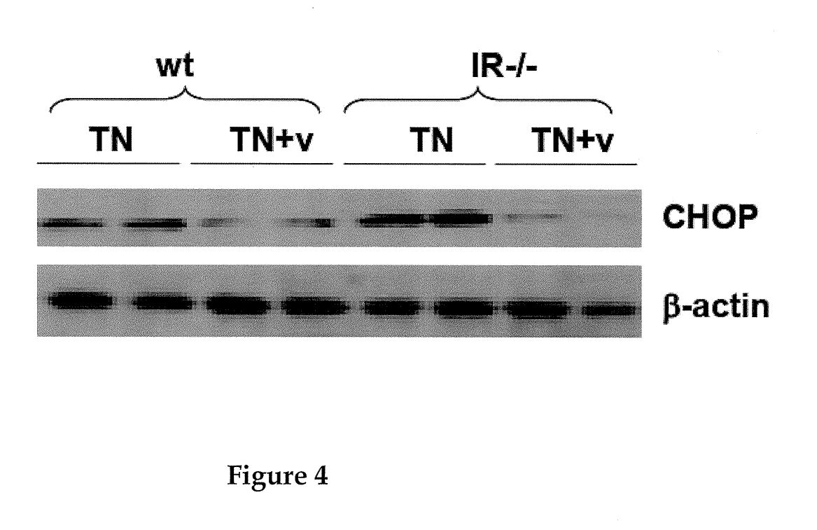

While ribozymes that cleave mRNA at site specific recognition sequences can be used to destroy target gene mRNAs, hammerhead ribozymes can be used. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Myers, 1995, Molecular Biology and Biotechnology: A Comprehensive Desk Reference, VCH Publishers, New York, (see especially FIG. 4, page 833) and in Haseloff & Gerlach, 1988, Nature, 334, 585-591, which is incorporated herein by reference in its entirety.

The ribozyme can be engineered so that the cleavage recognition site is located near the 5′ end of the target gene mRNA, i.e., to increase efficiency and minimize the intracellular accumulation of non-functional mRNA transcripts.

The ribozymes of the invention also include RNA endoribonucleases (hereinafter “Cech-type ribozymes”) such as the one that occurs naturally in Tetrahymena thermophila (known as the IVS, or L-19 IVS RNA) and that has been extensively described by Thomas Cech and collaborators (Zaug, et al., 1984, Science, 224, 574-578; Zaug and Cech, 1986, Science, 231, 470-475; Zaug, et al., 1986, Nature, 324, 429-433; published International patent application No. WO 88/04300 by University Patents Inc.; Been & Cech, 1986, Cell, 47, 207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in the target gene.

As in the antisense approach, the ribozymes can be composed of modified oligonucleotides (e.g., for improved stability, targeting, etc.) and should be delivered to cells that express the target gene in vivo. One method of delivery involves using a DNA construct “encoding” the ribozyme under the control of a strong constitutive pol III or pol II promoter, so that transfected cells will produce sufficient quantities of the ribozyme to destroy endogenous target gene messages and inhibit translation. Because ribozymes unlike antisense molecules, are catalytic, a lower intracellular concentration is required for efficiency.

Endogenous target gene expression can also be reduced by inactivating or “knocking out” the target gene or its promoter using targeted homologous recombination (e.g., see Smithies, et al., 1985, Nature 317, 230-234; Thomas & Capecchi, 1987, Cell 51, 503-512; Thompson, et al., 1989, Cell 5, 313-321; each of which is incorporated by reference herein in its entirety). For example, a mutant, non-functional target gene (or a completely unrelated DNA sequence) flanked by DNA homologous to the endogenous target gene (either the coding regions or regulatory regions of the target gene) can be used, with or without a selectable marker and/or a negative selectable marker, to transfect cells that express the target gene in vivo. Insertion of the DNA construct, via targeted homologous recombination, results in inactivation of the target gene. Such approaches are particularly suited modifications to ES (embryonic stem) cells can be used to generate animal offspring with an inactive target gene (e.g., see Thomas & Capecchi, 1987 and Thompson, 1989, supra). However this approach can be adapted for use in humans provided the recombinant DNA constructs are directly administered or targeted to the required site in vivo using appropriate viral vectors.

Alternatively, endogenous target gene expression can be reduced by targeting deoxyribonucleotide sequences complementary to the regulatory region of the target gene (i.e., the target gene promoter and/or enhancers) to form triple helical structures that prevent transcription of the target gene in target cells in the body. (See generally, Helene, 1991, Anticancer Drug Des., 6(6), 569-584; Helene, et al., 1992, Ann. N.Y. Acad. Sci., 660, 27-36; and Maher, 1992, Bioassays 14(12), 807-815).

Nucleic acid molecules to be used in triple helix formation for the inhibition of transcription should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC+ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

In instances wherein the antisense, ribozyme, and/or triple helix molecules described herein are utilized to inhibit mutant gene expression, it is possible that the technique may so efficiently reduce or inhibit the transcription (triple helix) and/or translation (antisense, ribozyme) of mRNA produced by normal target gene alleles that the possibility may arise wherein the concentration of normal target gene product present may be lower than is necessary for a normal phenotype. In such cases, to ensure that substantially normal levels of target gene activity are maintained, therefore, nucleic acid molecules that encode and express target gene polypeptides exhibiting normal target gene activity may, be introduced into cells via gene therapy methods such as those described, below, in Section 5.7.2 that do not contain sequences susceptible to whatever antisense, ribozyme, or triple helix treatments are being utilized. Alternatively, in instances whereby the target gene encodes an extracellular protein, the requisite level of target gene activity can be maintained by co-administering normal target gene protein.

Anti-sense RNA and DNA, ribozyme, and triple helix molecules of the invention may be prepared by any method known in the art for the synthesis of DNA and RNA molecules, as discussed above. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

The Function for Visfatin Provided by the Invention

Visfatin is a newly identified adipocytokine that is upregulated during obesity and exerts insulin-mimetic effects in peripheral tissues by binding to the insulin receptor. In the invention visfatin activity protects macrophages from ER-stress mediated apoptosis. Mechanistic studies indicate that visfatin directly targets distal UPR effectors without inhibiting upstream UPR activators. A distal UPR event, induction of the ATF4, is shown to be the direct target of visfatin. The mRNA level of ATF4 is intact, whereas the translation of ATF4 is halted in the presence of visfatin. The UPR-suppressing effect of visfatin is independent of the insulin receptor. Most significantly, administration of recombinant visfatin is found to suppress acute UPR induction in macrophages of the mouse peritoneal cavity, implying an anti-apoptotic role of visfatin in vivo.

Results from a model of FC-loaded macrophages can apply to a much broader spectrum of advanced lesional conditions, i.e., beyond FC accumulation, as lesions contain a number of ER stressors.

UPR Pathway

The UPR is a pathway responsive to conditions of ER stress. Normally, proteins that require modification for proper function are trafficked to the ER at which point their further processing is regulated by a dedicated protein maturation machinery. It is within this organelle that proteins adopt their correct three dimension fold through the action of ER molecular chaperones. Further modifications, such as glycosylation and disulfide bond formation also occur within the ER.

Specific conditions of stress, such as an increased demand for protein production, can overwhelm the ER resident processing machinery and result in the activation of the UPR pathway as a compensatory mechanism to reduce the ER burden. The UPR response is a multi-phasic process that encompasses four discernible mechanisms including, translational attenuation, enhanced expression of ER chaperones, enhanced expression of ER-associated degradation (ERAD) components and the induction of apoptosis.

Conditions of ER stress and detected by transmembrane proteins located in the ER. PRKR-like endoplasmic reticulum kinase (PERK) is a type I transmembrane involved in the detection of unfolded proteins within the lumen of the ER. In the absence of stress, binding of BiP to the luminal domain of PERK maintains the transmembrane receptors in an inactive state. When misfolded proteins accumulate with the ER, BiP dissociates from PERK and binds preferentially to the misfolded client proteins. Upon release of BiP inhibition, PERK molecules oligomerize and undergo activation via transphosphorylation. Activation of PERK, in turn results in the phosphorylation-dependent inactivation of eukaryotic translation initiation factor (eIF2alpha). Attenuation of eIF2alpha activity acts as a signal to promote translation of the ER-stress responsive transcription factor ATF4. The targets of ATF4 transcriptional activity comprise the stress responsive transcription factor ATF3, the pro-apoptotic transcription factor CHOP/Gadd153 (C/EBP homology protein) as well as several antioxidant genes and genes encoding ER protein maturation machinery. Upregulation of antioxidant genes is an important component of the UPR response as the formation of disulfide bonds in the ER is a reactive oxygen species (ROS) generating phenomenon. CHOP, in turn, activates the transcription factor GADD34, ERO1 (an ER oxidase), DR5 (Death Receptor 5) and carbonic anhydrase VI. GADD34 association with protein phosphate 2 enhances dephosphorylation of eIF2alpha and promotes ER client protein biosynthesis.

In addition to PERK, two other ER membrane resident proteins have been implicated the ER stress response. The first of these, ATF6, is a transmembrane type II protein consisting of a BiP binding luminal domain and a cytoplasmic domain that has a basic-leucine zipper motif DNA binding domain and a transactivation domain. When unfolded proteins accumulate in the ER, BiP dissociates from ATF6 and ATF6 is translocated to the Golgi apparatus at which point it is cleaved to generate pATF6(N). This cytosolic fragment then translocates to the nucleus where it activates transcription of ER chaperone genes such as BiP, GRP94 and calreticulin. The second of these ER membrane resident stress responsive sensors is IRE1. Upon ER stress, BiP dissociates from luminal domain of IRE1 at which point IRE1 proteins undergo a process of oligomerization-induced transactivation. Upon activation, the RNase domain on the cytosolic domain of IRE1 converts XBP1 (x-box binding protein 1) pre-mRNA into mature mRNA by a mechanisms of non-conventional splicing to produce the transcription factor pXBP1(S). pXBP1(S) in turn activates the transcription of genes involved in the ERAD response such as; EDEM, HRD1, Derlin-2 and Derlin-3 gene encoding ER chaperones such as, BiP, p581PK, ERdj4, PDI-P5 and HEDJ.

UPR and Apoptosis

A number of molecules, including CHOP, Bax/Bak, Caspase 12 and IRE are important regulators of UPR-induced apoptosis. ER-stressed induced activation of CHOP results in the suppression of expression of the anti-apoptotic factor Bcl2 and subsequently further release of calcium from the ER lumen by a Bak and Bax related process. Another, and possibly related, pathway involves the recruitment of the tumor necrosis factor (TNF) receptor associated factor (TRAF)₂ to activated IRE1, which in turn results in the activation of caspase 12 and c-jun N-terminal kinase (JNK) signaling. Further downstream events may comprise the activation of caspase 9 and 7 and NF-kappaB activation. Apoptosis signal-regulating kinase 1 (ASK1) signaling may function upstream of JNK activation in this process.

Atherosclerosis

Atherosclerosis is a chronic immune inflammatory disease which is initiated by interaction of activated luminal endothelium. This localized activation can occur for a variety of reasons, including endothelial damage or stress. Upon attachment to the endothelial surface, monocytes migrate into the subendothelial space where they differentiate into macrophages. The newly formed macrophages then begin to ingest a variety of atherogenic lipoproteins in the subendothelial space and accumulate significant amounts of lipids in their cytoplasm and transform into foam cells. Many of these atherogenic factors are lipoproteins that have been modified by both enzymatic and non-enzymatic processes within the arterial intima. The lipid laden foam cells in turn aggregate into a atheromatous core and undergo programmed cell death. This necrotic core consists largely of lipids, aggregated cholesterol and cell debris.

Cholesterol Uptake

Once in the arterial intima, macrophages can ingest cholesterol via several different internalization pathways. Scavenger receptors, such as SR-A or CD36 actively internalize oxidativley modified lipoproteins, such as oxidized-LDL (ox-LDL). SR-BI is antoher scavenger receptor expressed in foam cells that has the ability to bind to both unmodified and modified lipoproteins. Another multi-ligand receptor is LRP1 (LDL receptor related protein), which functions in the uptake of apoE lipoproteins, as well as other members of the LDL receptor family, including LDLR, have been implicated in cholesterol uptake by macrophages. Fluid uptake by micropinocytosis and macropinocytosis can also contribute to this process.

Cholesterol Homeostatis

Macrophages are not typically effective at limiting the rate of cholesterol intake at the level of receptor internalization and mechanisms to regulate intracellular cholesterol homeostasis depend in large part on processes that promote cholesterol efflux or cholesterol storage in intracellular compartments. ATP-binding cassette transporters, such as ABCA1 and ABCG1, are transmembrane proteins that function to transport a variety of substrates, including cholesterol, across cellular membranes in an energy-dependent manner. Any excess amount of cholesterol that is not effluxed by these pathways is stored in the form of cholesteryl esters in membrane bound cytoplasmic lipid droplets. Esterification occurs in the ER by the ER-resident enzymes called acyl-coenzyme A:cholesterol acyl transferases (ACATs).

Macrophages Death Induced by Cholesterol Accumulation

The uptake of native and modified cholesterol by macrophages has a dual consequence depending on the stage and progression of an atherosclerotic lesion. In early stages, the ingestion of cholesterols and other lipids can have a protective effect by removing these cytotoxic and pro-inflammatory molecules form the extracellular environment. However, in later stages, as macrophages become lipid-laden foam cells and aggregate into atherosclerotic clusters, the cells undergo programmed cell death resulting from excessive intracellular levels of cholesterol and form an atheromatous necrotic core bounded by a fibrous cap to create a localized plaque. The eventual rupture of atheromatous plaques can result in stenosis (narrowing of the vessel) or thrombosis and infarction (i.e. myocardial infarction—heart attack).

Cholesterol Induced Activation of UPR

Under normal conditions, the process of cholesterol efflux and cholesterol esterification is sufficient to maintain intracellular cholesterol homeostasis. Under conditions of excessive cholesterol uptake, such as those encountered by macrophages in atherosclerotic lesions, these process cannot compensate for the increased levels and cholesterol is redistributed to about intracellular membranes. Free cholesterol (FC) has the ability to insert into lipid bilayers and change the physical properties of biological membranes. Normally present at low concentrations in the ER membrane, the accumulation of excessive levels of FC in the ER causes an activation of the UPR response by inducing a depletion of ER-resident calcium stores. Activation of the UPR pathway can eventually result in programmed cell death. In macrophages, apoptosis caused by ER stress and activation of the UPR in response to cholesterol accumulation occurs through CHOP induction and the synergistic activation of signaling downstream of p38 MAPK, the SRA receptor and c-Jun Nh2-termial kinase (JNK)₂.

Visfatin

Initially termed Pre-B Cell Colony Enhancing Factor (PBEF) on the basis of its ability to synergize with IL-7 to promote the differentiation of B-cell precursors, Visfatin is now classified as an adipokine that promotes glucose mobilization in adipocytes, myocytes and hepatocytes. Predominantly expressed in adipose tissues, visfatin is also expressed in bone marrow, skeletal muscle and liver among other cell types and is a protein hormone secreted into circulation in people with obesity. Visfatin also stimulates the differentiation of preadipocytes to mature fat cells, induces triglyceride accumulation, accelerates triglyceride synthesis from glucose, and induces the expression of genes encoding the adipose tissue-specific markers peroxisome proliferator-activated receptor-g (PPARg), fatty acid synthase, diacylglycerol acyltransferase, and adiponectin. Circulating levels of visfatin have also been correlated to obesity. The effects of visfatin on glucose metabolism may be related in part to its ability to bind the insulin receptor and activate downstream signaling events. Binding to the insulin receptor occurs at a site that is independent of insulin binding. This adipokine is also upregulated by IL-1b and functions as an inhibitor of apoptosis in neutrophils and muscle cells. Visfatin also increases IL-6 and IL-8 secretion. In addition to proposed a proposed intracellular function in regulating NAD+ biosynthesis, visfatin has been implicated in atherosclerosis. White adipose tissue-derived macrophages produce visfatin at detectable levels and its expression enhanced in lipid loaded macrophages and in atherosclerotic carotid plaques. Visfatin expression is also stimulated by dexamethasone, peroxisome proliferator-activated receptor agonists and TFN-alpha.

### EMBODIMENTS OF THE INVENTION

In specific embodiments, a visfatin polypeptide, a derivative of a visfatin polypeptide, an analog of a visfatin polypeptide, a peptidomimetic agent of a visfatin polypeptide, a truncation product of a visfatin polypeptide, a fragment of a visfatin polypeptide, a polypeptide that is homologous to visfatin can be used to increase visfatin activity-induced phagocyte survival associated with advanced atherosclerotic lesions. In other embodiments, a nucleic acid molecule that can encode a visfatin polypeptide, an analog of a visfatin polypeptide, a peptidomimetic agent of a visfatin polypeptide, a truncation product of a visfatin polypeptide, a fragment of a visfatin polypeptide, a polypeptide that is homologous to visfatin or a polypeptide can be used to increase visfatin activity-induced phagocyte survival associated with advanced atherosclerotic lesions.

In one embodiment, the compound used to increase visfatin activity-induced phagocyte survival associated with advanced atherosclerotic lesions is a visfatin polypeptide. In another embodiment, the compound used to increase visfatin activity-induced phagocyte survival associated with advanced atherosclerotic lesions is a fragment of a visfatin polypeptide. In yet another embodiment, the compound used to increase visfatin activity-induced phagocyte survival associated with advanced atherosclerotic lesions is a nucleic acid molecule capable of encoding a visfatin polypeptide.

Combinations of compounds can be used to prevent or treat atherosclerosis using at least one compound described herein or identified using methods described herein. Such combinations can comprise, e.g., two or more compounds that increase visfatin activity-induced phagocyte survival associated with advanced atherosclerotic lesions or at least one compound that increase visfatin activity-induced phagocyte survival associated with advanced atherosclerotic lesions and at least one compound useful for treating atherosclerosis whose method of function is unknown or does not directly relate to increasing visfatin activity-induced phagocyte survival associated with advanced atherosclerotic lesions.

In one aspect, the invention provides for a pharmaceutical composition comprising a compound that increases visfatin activity-induced phagocyte survival associated with advanced atherosclerotic lesions and a compound that reduces cholesterol ingestion by phagocytes. In one embodiment, the combination comprises visfatin and a compound that can act as an inhibitor of cholesterol uptake by phagocytes associated with advanced atherosclerotic lesions. In another embodiment, the combination comprises visfatin and an acyl-coenzyme A cholesterol acyltransferase (ACAT) inhibitor.

In one aspect, the invention provides for a pharmaceutical composition comprising a compound that increases visfatin activity-induced phagocyte survival associated with advanced atherosclerotic lesions and a compound that is capable of activating Stat3. In one embodiment, the combination comprises visfatin and IL-10. In another embodiment, the combination comprises visfatin and VEGF.

The compounds described herein can be used in the preparation of a medicament for use in the treatment of atherosclerosis, e.g., atherosclerosis associated with advanced atherosclerotic lesions that can be ameliorated using a compound that increases visfatin activity with such lesions.

In one aspect, the invention provides a method for treating vascular disease in a subject by administering to the subject a pharmaceutically effective amount of a compound to increase visfatin activity to suppress cell death resulting from UPR pathway activation. In another aspect, the invention provides a method for inhibiting the development of vascular disease in a subject by administering to the subject a pharmaceutically effective amount of a compound to increase visfatin activity to suppress cell death resulting from UPR pathway activation. In yet another aspect, the invention provides a method for treating a subject at risk of developing a vascular disease by administering to the subject a pharmaceutically effective amount of a compound to increase visfatin activity to suppress cell death resulting from UPR pathway activation.

In one embodiment, a visfatin polypeptide is administered to the subject to increase visfatin activity in the subject.

### EXAMPLES

### Example 1

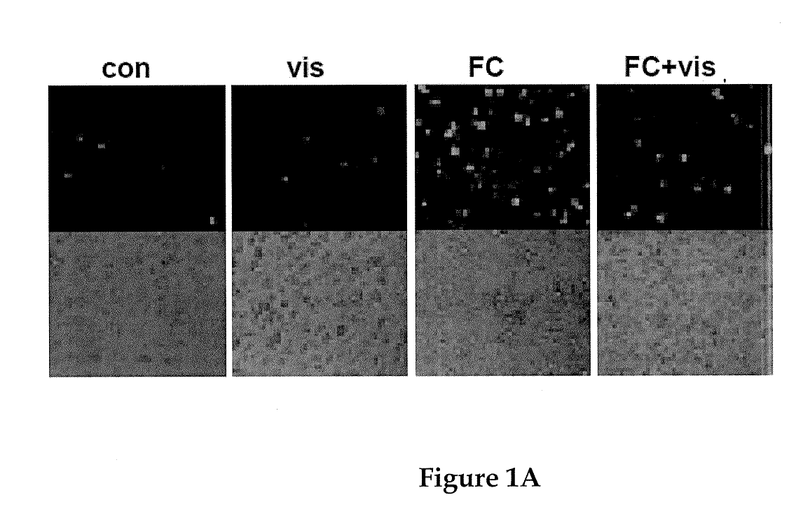

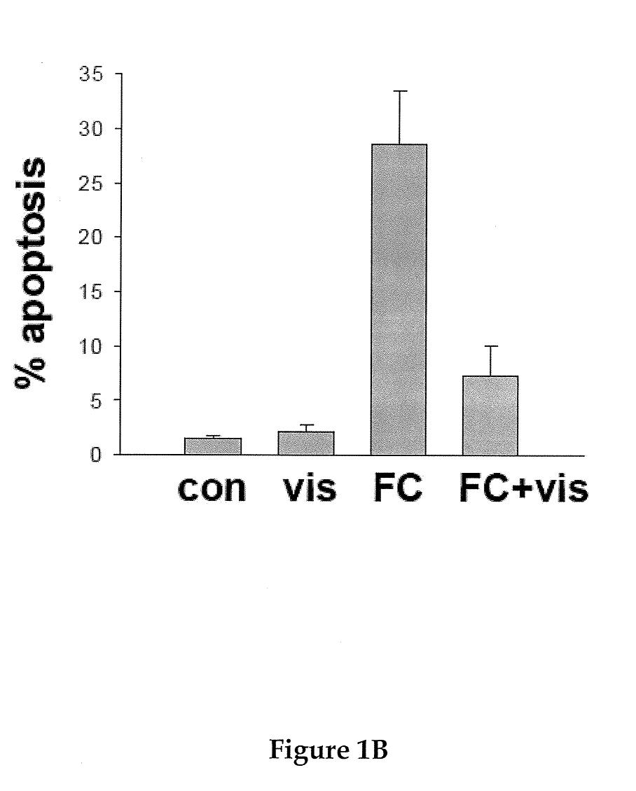

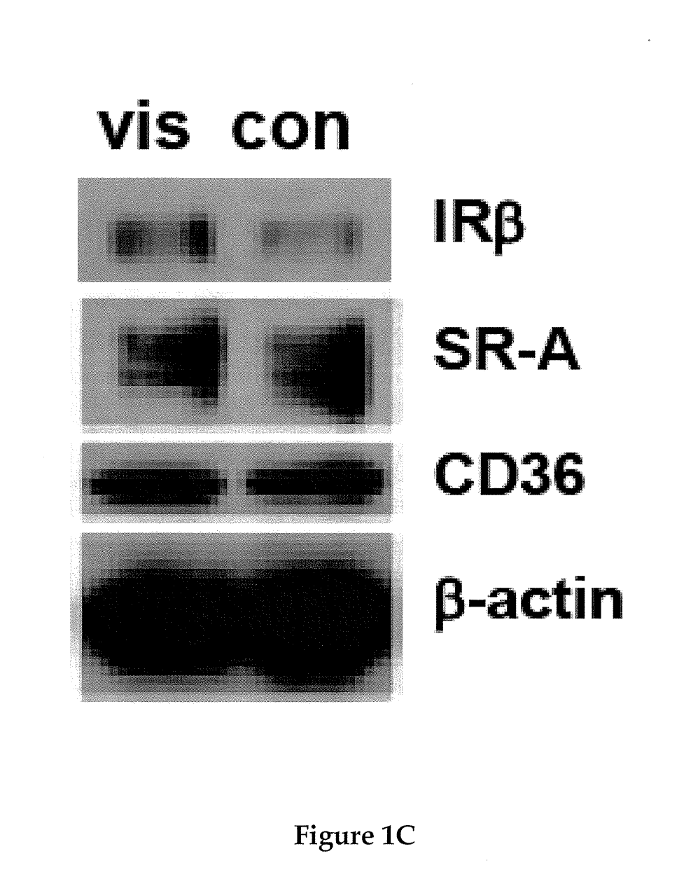

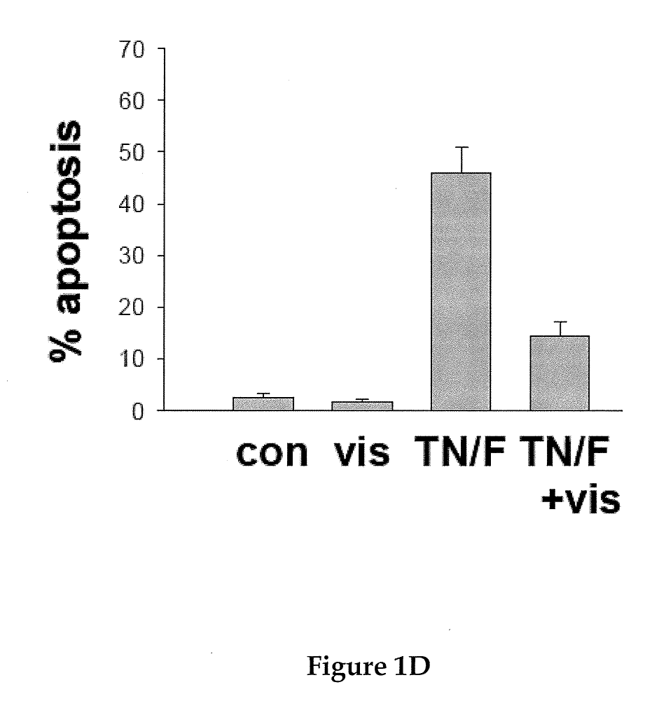

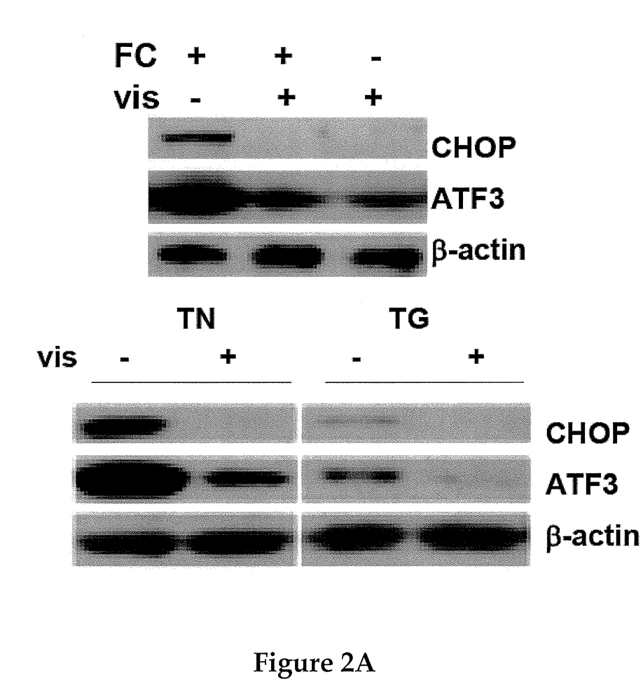

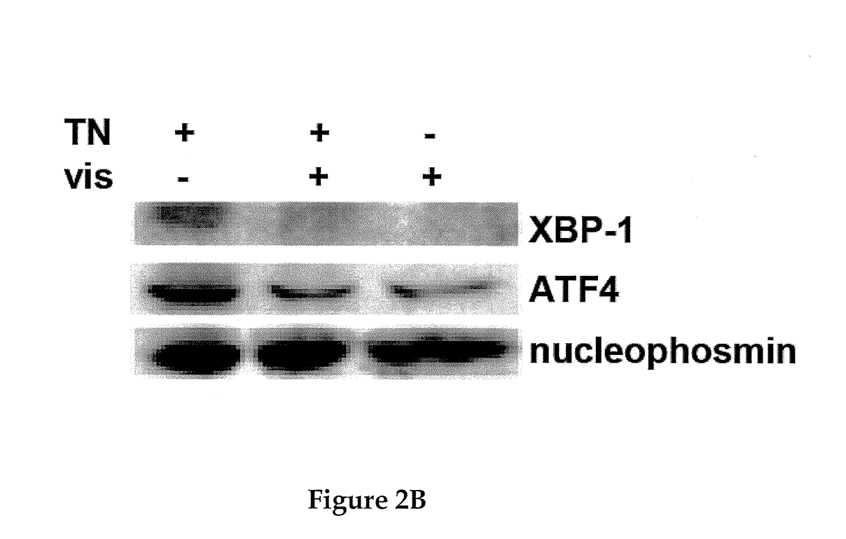

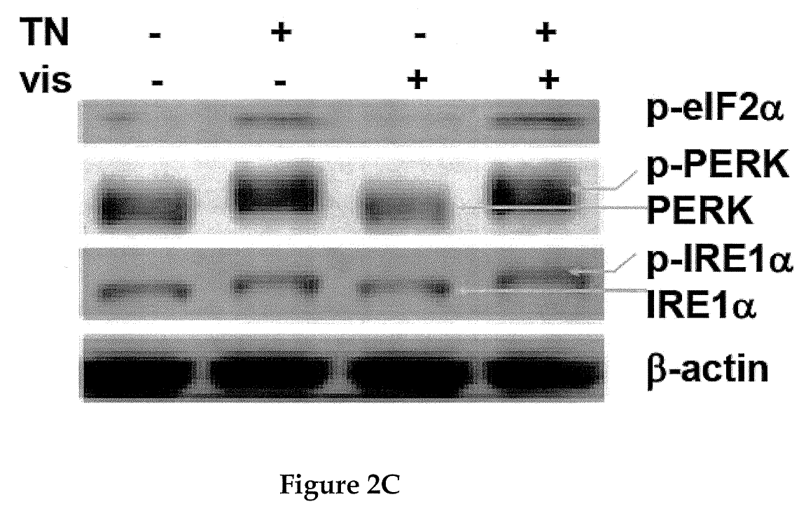

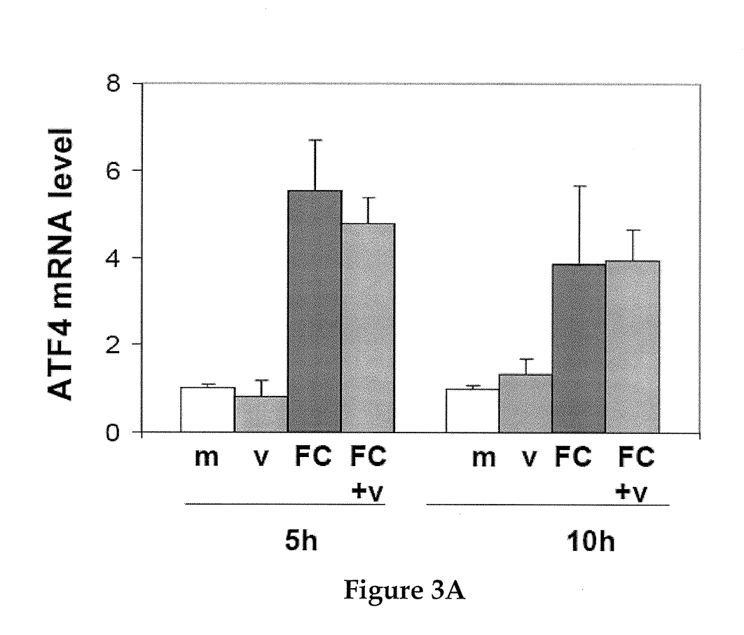

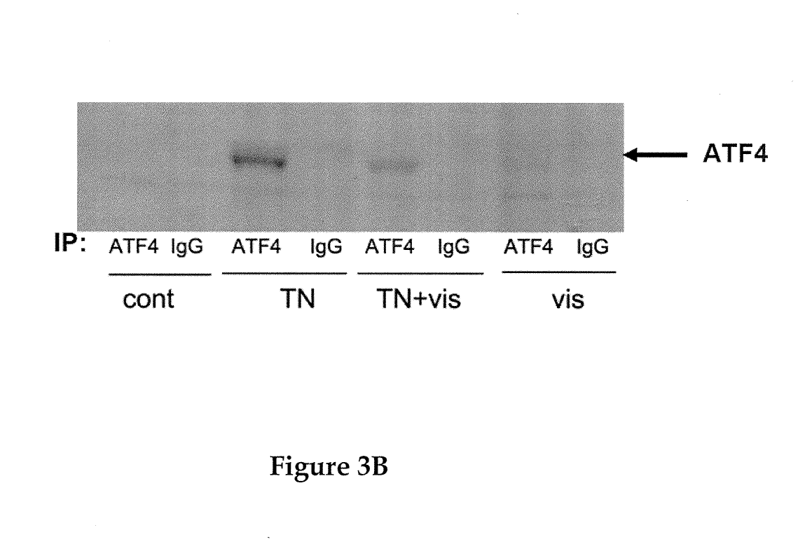

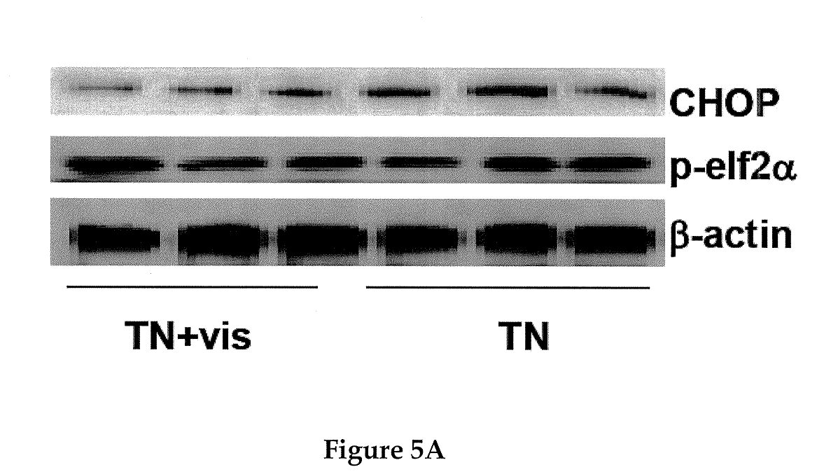

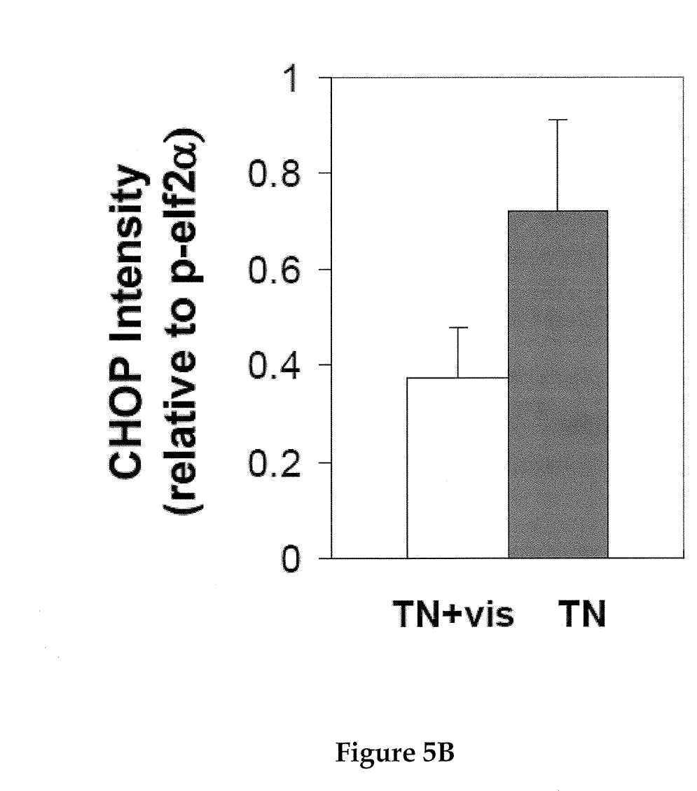

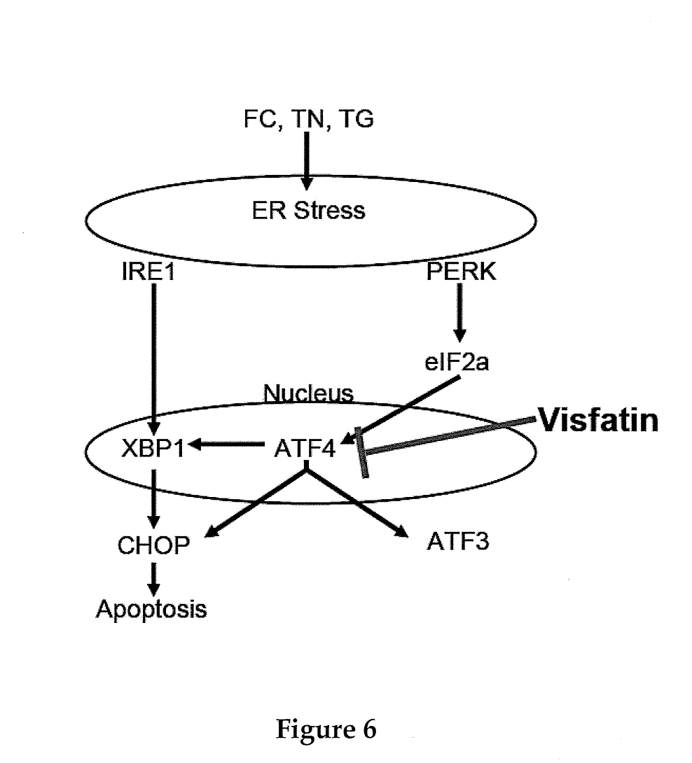

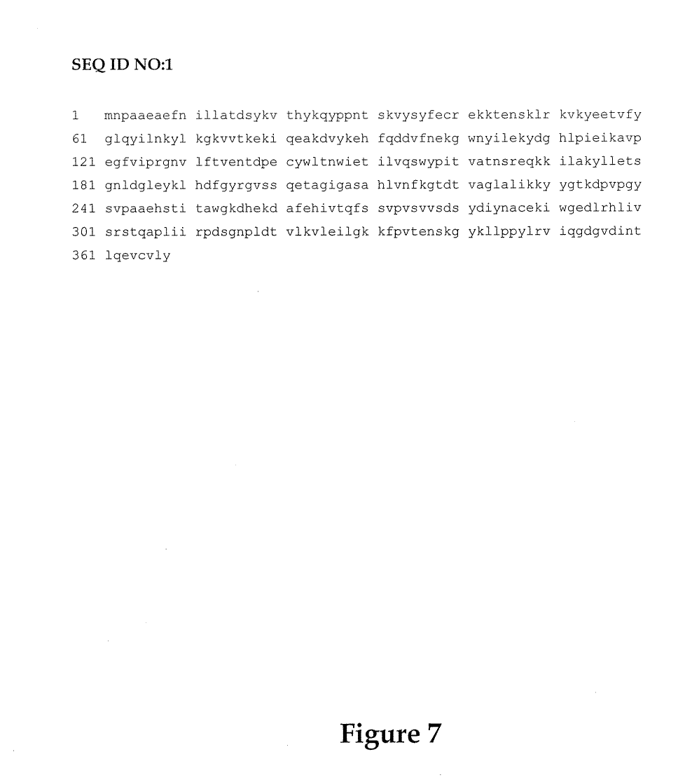

In advanced atherosclerotic lesions, accumulation-of large amounts of free cholesterol (FC) within lesional macrophages induces macrophage apoptosis, which is speculated to contribute to plaque instability. A key event in FC-induced apoptosis is the activation an ER stress signaling pathway named the unfolded protein response (UPR). Results from a model of FC-loaded macrophages likely apply to a much broader spectrum of advanced lesional conditions, i.e., beyond FC accumulation, as lesions contain a number of ER stressors. Visfatin is a newly identified adipocytokine that is upregulated during obesity and exerts insulin-mimetic effects in peripheral tissues by binding to the insulin receptor. In the present study, we show that visfatin protects macrophages from ER-stress mediated apoptosis (FIG. 1). Mechanistic studies indicate that visfatin directly targets distal UPR effectors without inhibiting upstream UPR activators (FIG. 2). A distal UPR event, induction of the ATF4, is shown to be the direct target of visfatin (FIG. 3). The mRNA level of ATF4 is intact, whereas the translation of ATF4 is halted in the presence of visfatin. The UPR-suppressing effect of visfatin is independent of the insulin receptor (FIG. 4). Most significantly, administration of recombinant visfatin is found to suppress acute UPR induction in macrophages of the mouse peritoneal cavity, implying an anti-apoptotic role of visfatin in vivo (FIG. 5).

### Example 2

Recombinant visfatin polypeptides will be injected into mice susceptible to atherosclerotic plaque formation. In accordance with this prophetic example, the genome of these mice can comprise a transgenic modification wherein ApoE1 gene expression is reduced to levels that promote atherosclerosis. The mice will be continuously fed a high cholesterol diet to promote the formation of atherosclerotic lesions. One group of mice will be administered a pharmaceutically effective amount of visfatin polypeptide in a suitable diluent and another group of mice will be administered diluent alone and the incidence of atherosclerotic plaque formation will be compared between the two groups. The amount of visfatin polypeptide, the length of administration and the frequency of administration are variables that can readily be determine by an individual skilled in the art. The example will show that the group of mice treated with visfatin have a lower incidence of atherosclerotic plaque formation than the group of mice administered the diluent alone.

### Example 3

Recombinant visfatin polypeptides will be injected into mice susceptible to atherosclerotic plaque formation. In accordance with this prophetic example, the genome of these mice can comprise a transgenic modification wherein ApoE1 gene expression is reduced to levels that promote atherosclerosis. The mice will be continuously fed a high cholesterol diet to promote the formation of atherosclerotic lesions. One group of mice will be administered a pharmaceutically effective amount of visfatin polypeptide in a suitable diluent and another group of mice will be administered diluent alone and the amount of macrophage death in atherosclerotic plaques will be compared between the two groups. The amount of visfatin polypeptide, the length of administration and the frequency of administration are variables that can readily be determine by an individual skilled in the art. The example will show that macrophages in atherosclerotic plaques in the group of mice treated with visfatin have a lower amount of cell death than macrophages in atherosclerotic plaques in the group of mice administered the diluent alone.

### Example 4

Recombinant visfatin polypeptides will be injected into mice susceptible to an ER-stress related disease. In accordance with this prophetic example, the genome of these mice can comprise a transgenic modification that causes an ER-stress related disease. Alternatively, the mice can be subjected to treatments that cause an ER stress related disease. The ER stress related disease can be selected from the group comprising: Alzheimer's disease, Parkinson's disease, Huntington's disease, spinobulbar muscular atrophy (Kennedy disease), Machado-Joseph disease, dentatorubral-pallidoluysian disease (Haw River Syndrome), spinocerebellar ataxia, Pelizaeus-Merzbacher disease, Prion disease, Creutzfeldt-Jakob disease, Gertsmann-Straussler-Scheinker syndrome, fatal familial insomnia, Kuru, Alpers syndrome, bovine spongiform encephalopathy, transmissible milk encephalopathy, chronic wasting disease, scrapie, amyotrophic lateral sclerosis (Lou Gehrig's disease), GM1 gangliosidosis, bipolar disorders, type I diabetes mellitus, type II diabetes mellitus, Walcott-Rallison syndrome or hereditary tyrosinemia type I, or any combination thereof. One group of mice will be administered a pharmaceutically effective amount of visfatin polypeptide in a suitable diluent and another group of mice will be administered diluent alone and the amount of ER stress related disease in will be compared between the two groups. The amount of visfatin polypeptide, the length of administration and the frequency of administration are variables that can readily be determine by an individual skilled in the art. The example will show that the group of mice treated with visfatin have a lower incidence of the ER stress related disease than the group of mice administered the diluent alone.

### BIBLIOGRAPHY

Misra U K, Pizzo S V. Up-regulation of GRP78 and antiapoptotic signaling in murine peritoneal macrophages exposed to insulin. J Leukoc Biol. 2005 July; 78(1):187-94. Epub 2005 Apr. 21.
Feng B, Yao P M, Li Y, Devlin C M, Zhang D, Harding H P, Sweeney M, Rong J X, Kuriakose G, Fisher E A, Marks A R, Ron D, Tabas I. The endoplasmic reticulum is the site of cholesterol-induced cytotoxicity in macrophages. Nat Cell Biol. 2003 September; 5(9):781-92. Epub 2003 Aug. 10.
Pedruzzi E, Guichard C, Ollivier V, Driss F, Fay M, Prunet C, Marie J C, Pouzet C, Samadi M, Elbim C, O'dowd Y, Bens M, Vandewalle A, Gougerot-Pocidalo M A, Lizard G, Ogier-Denis E. NAD(P)H oxidase Nox-4 mediates 7-ketocholesterol-induced endoplasmic reticulum stress and apoptosis in human aortic smooth muscle cells. Mol Cell Biol. 2004 December; 24(24):10703-17.
Devries-Seimon T, Li Y, Yao P M, Stone E, Wang Y, Davis R J, Flavell R, Tabas I. Cholesterol-induced macrophage apoptosis requires ER stress pathways and engagement of the type A scavenger receptor. J Cell Biol. 2005 Oct. 10; 171(1):61-73. Epub 2005 Oct. 3.
Han S, Liang C P, DeVries-Seimon T, Ranalletta M, Welch C L, Collins-Fletcher K, Accili D, Tabas I, Tall A R. Macrophage insulin receptor deficiency increases ER stress-induced apoptosis and necrotic core formation in advanced atherosclerotic lesions. Cell Metab. 2006 April; 3(4):257-66.

1-78. (canceled)

79. A method for treating a subject having, or at risk of having a vascular disease or an ER stress-related disease, the method comprising administering to the subject a pharmaceutically effective amount of a visfatin polypeptide or a visfatin nucleic acid to increase visfatin activity.

80. The method of claim 79, wherein visfatin activity comprises suppression of unfolded protein response (UPR) pathway activation, ERK activation, AKT activation, protection of phagocytes from endoplasmic reticulum (ER) stress mediated cell death, suppression of UPR activation induced production of CHOP, suppression of UPR activation induced production of ATF3, suppression of UPR activation induced production of ATF4, suppression of UPR activation induced production of XBP1, or activation of insulin receptor signaling, or any combination thereof.

81. The method of claim 79, wherein the visfatin polypeptide comprises a peptidomimetic, a truncated visfatin polypeptide that exhibits visfatin activity, a fragment of a visfatin polypeptide that exhibits visfatin activity, a polypeptide of SEQ ID NO:1, or a polypeptide having a sequence at least 85% identical to the amino acid sequence in SEQ ID NO:1 that exhibits visfatin activity.

82. The method of claim 79, wherein the visfatin nucleic acid comprises a nucleic acid molecule that encodes a truncated visfatin polypeptide that exhibits visfatin activity, a nucleic acid molecule that encodes a fragment of a visfatin polypeptide that exhibits visfatin activity, a nucleic acid molecule that encodes a polypeptide of SEQ ID NO:1, or a nucleic acid molecule that encodes polypeptide having a sequence at least 85% identical to the amino acid sequence in SEQ ID NO:1 that exhibits visfatin activity.

83. The method of claim 79, wherein the visfatin polypeptide has at least 99%, 97%, 95%, 90%, 80% or 70% amino acid sequence identity to the amino acid sequence in SEQ ID NO:1.

84. The method of claim 79, wherein the nucleic acid molecule encoding a visfatin polypeptide has at least 99%, 97%, 95%, 90%, 80% or 70% amino acid sequence identity to the amino acid sequence in SEQ ID NO:1.

85. The method of claim 79, wherein the subject has a necrotic core in an atherosclerotic plaque.

86. The method of claim 85, wherein the necrotic core comprises dead phagocytes.

87. The method of claim 79, wherein the subject has impaired phagocyte function.

88. The method of claim 87, wherein the impaired phagocyte function is caused by activation of the UPR pathway.

89. The method of claim 88, wherein the activation of the UPR pathway is caused by ER stress, enrichment of free cholesterol in the ER membranes of the phagocyte, exposure of the phagocyte to a condition of hypoxia, uptake of excessive cholesterol by the phagocyte, uptake of excessive oxidized LDL by the phagocyte, uptake of excessive acetylated LDL by the phagocyte, reduced cholesterol esterification in the phagocyte, conversion of the phagocyte to a foam cell, or any combination thereof.

90. The method of claim 79, wherein the vascular disease comprises atherosclerosis, arteriosclerosis, thrombosis, restenosis, hypertension, angina pectoris, arrhythmia, embolism, stroke, heart failure, myocardial infarction, thrombosis, thromboembolysis, peripheral vascular disease, cerebral ischemia, or cardiomyopathy, hypercholesterolemia, hyperlipoproteinemia, hypertriglyceridemia, lipodystrophy, hyperglycemia, reduced HDL levels, elevated LDL levels, low glucose tolerance, insulin resistance, obesity, dyslipidemia, hyperlipidemia, hypercholesterolemia, Type I diabetes, Type II diabetes, hyperinsulinemia, atherogenesis, aneurysm, ischemia, coronary plaque inflammation, arrhythmia or any combination thereof.

91. The method of any of claim 86 or 87, wherein the phagocyte comprises a microglial cell, a monocyte, a microglial precursor cell, a monocyte precursor cell, a macrophage precursor cell, a microglial-like cell, a monocyte-like cell, a dendritic-like cell, a macrophage, or a macrophage-like cell.

92. The method of claim 79, wherein the method further comprises administering one or more therapeutic agents.

93. The method of claim 92, wherein the therapeutic agent inhibits UPR-induced cell death.

94. The method of claim 93, wherein the therapeutic agent comprises a p38 MAPK inhibitor, a p38 substrate peptide, a JNK2 inhibitor, an SRA inhibitor, a lipoxin, a lipoxin analog, a compound that stimulates lipoxin synthesis or activity, a statin, a beta-blocker, a thiozide diuretic, an angiotensin-converting enzyme inhibitor, an omega-3 fatty acid, aspirin, a clopidogrel, an aldosterone agonist, a nitrate, a calcium channel blocker, a cholesterol-uptake inhibitor, a cholesterol biosynthesis inhibitor, an HMG-CoA synthase inhibitor, a squalene epoxidase inhibitor, a squalene synthetase inhibitor, an acyl-coenzyme A cholesterol acyltransferase (ACAT) inhibitor, a niacinamide, a cholesterol absorption inhibitor, a fibrate, vitamin B6, vitamin B12, vitamin B3, an anti-oxidant vitamin, an angiotensin II receptor antagonist, a renin inhibitor, a platelet aggregation inhibitor, ethyl icosapentate, amlodipine, U18666A, celecoxib, an anti-inflammatory agent an anti-arrhythmic agent or any combination thereof.

95. The method of claim 94, wherein the p38 MAPK inhibitor comprises SB202190, PD169316, FR167653, SB203580, ARRY-797, SB 239063, SC-68376, SB 220025, SB-200646, PD 169316, or SKF-86002.

96. The method of claim 94, wherein the JNK2 inhibitor comprises SP600125, a polypeptide comprising residues 153-163 of JNK-interacting protein-1 (JIP-1), AS601245, or N-(4-Amino-5-cyano-6-ethoxypyridin-2-yl)-2-(2,5-dimethoxyphenyl)acetamide.

97. The method of claim 94, wherein the SRA inhibitor comprises an SRA blocking antibody.

98. The method of claim 94, wherein the cholesterol biosynthesis inhibitor comprises an HMG-CoA reductase inhibitor or a statin.

99. The method of claim 94, wherein the acyl-coenzyme A cholesterol acyltransferase (ACAT) inhibitor comprises melinamide, probucol, 58035, or nicotinic acid.

100. The method of claim 94, wherein the cholesterol absorption inhibitor comprises beta-sitosterol or ezetimibe.

101. The method of claim 94, wherein the fibrate comprises clofibrate, bezafibrate, fenofibrate, or gemfibrozil.

102. The method of claim 94, wherein the vitamin B12 comprises cyanocobalamin or hydroxocobalamin.

103. The method of claim 94, wherein the anti-oxidant vitamin comprises vitamin C, vitamin E, or betacarotene.

104. The method of claim 94, wherein the platelet aggregation inhibitor comprises a fibrinogen receptor antagonist.

105. The method of claim 94, wherein the therapeutic agent comprises Interleukin-1, Interleukin-10, Interleukin-6, Interleukin-22, Vascular Endothelial Growth Factor, leptin, basic Fibroblast Growth Factor, Leukemia Inhibitory Factor, Epidermal Growth Factor, Neuregulin-1, Growth Hormone, Interleukin-4, Ciliary Neurotrophic Factor, or Proteolysis-Inducing Factor or any combination thereof.

106. The method of claim 79, wherein the ER stress-related disease comprises Alzheimer's disease, Parkinson's disease, Huntington's disease, spinobulbar muscular atrophy (Kennedy disease), Machado-Joseph disease, dentatorubral-pallidoluysian disease (Haw River Syndrome), spinocerebellar ataxia, Pelizaeus-Merzbacher disease, Prion disease, Creutzfeldt-Jakob disease, Gertsmann-Straussler-Scheinker syndrome, fatal familial insomnia, Kuru, Alpers syndrome, bovine spongiform encephalopathy, transmissible milk encephalopathy, chronic wasting disease, scrapie, amyotrophic lateral sclerosis (Lou Gehrig's disease), GM1 angliosidosis, bipolar disorders, type I diabetes mellitus, type II diabetes mellitus, Walcott-Rallison syndrome or hereditary tyrosinemia type I, or any combination thereof.

107. A pharmaceutical composition comprising a visfatin polypeptide and a pharmaceutically acceptable adjuvant, diluent or carrier.

108. The composition of claim 107, wherein the composition further comprises (i) a cholesterol-lowering agent, (ii) a beta blocker or (iii) a anti-inflammatory agent.
